(19) United States
(12) Patent Application Publication
(10) Pub. No.: US 2020/0075146 A1
(43) Pub. Date: Mar. 5, 2020

(54) CONSULTATION SUPPORT SYSTEM

(71) Applicant: Canon Medical Systems Corporation, Otawara-shi (JP)

(72) Inventors: Yosuke Yanagida, Nasushiobara (JP); Tomoya Takefuji, Utsunomiya (JP)

(73) Assignee: Canon Medical Systems Corporation, Otawara-shi (JP)

(21) Appl. No.: 16/554,875

(22) Filed: Aug. 29, 2019

(30) Foreign Application Priority Data
Aug. 31, 2018 (JP) 2018-163583
Aug. 27, 2019 (JP) 2019-154990

(51) Int. Cl. G16H 15/00; G16H 10/60; G16H 50/20

(52) U.S. Cl. CPC G16H 15/00; G16H 50/20; G16H 10/60

(57) ABSTRACT

According to one embodiment, a consultation support system includes processing circuitry. The processing circuitry is configured to extract information that specifies or suggests a type of an abnormality in a result of an examination concerning an object from an analysis result obtained by analyzing the result of the examination, decide a handling destination capable of making a consultation about the abnormality specified or suggested in the analysis result using the extracted information that specifies or suggests the type of the abnormality, and display information concerning a referral letter to the decided handling destination on a display if a request source that has requested the examination is not included in the decided handling destination.

### CROSS-REFERENCE TO RELATED APPLICATIONS

This application is based upon and claims the benefit of priority from the prior Japanese Patent Applications No. 2018-163583, filed Aug. 31, 2018; and No. 2019-154990, filed Aug. 27, 2019, the entire contents of all of which are incorporated herein by reference.

### FIELD

Embodiments described herein relate generally to a consultation support system.

### BACKGROUND

Various kinds of examinations such as specimen examinations and physiological function examinations are conducted for an object based on a request from a medical department. Examples of the specimen examinations are hematological examinations and biochemical examinations. In addition, examples of the physiological function examinations are radiological examinations (for example, X-ray CT (Computed Tomography) imaging, x-ray imaging, MR (Magnetic Resonance) imaging, ultrasonic diagnosis, and the like), cardiac examinations (for example, electrocardiogram, phonocardiogram, pulse wave, and the like), electroencephalography, fundoscopy, and respiratory function tests.

Various kinds of examinations are executed according to a request from a medical department, and the examination results are returned to the medical department in a form of reports (diagnostic reports). Conventionally, there exist following systems that electronically support these examinations.
A system that issues an examination request to a medical department that is in charge of an examination by an HIS (Hospital Information System, including electronic medical records).
A system/software (for example, an RIS (Radiology Information System) in radiology) that manages an examination appointment in a medical department that is in charge of an examination.
A system/software that generates/manages a report for an examination result.

A system/software that notifies an electronic medical record that a report is generated.

A medical department, needs to appropriately respond in accordance with the contents of a report. In some cases, a report points out a disease to be handled by a medical department (to be referred to as “another medical department” hereinafter) specializing in the disease, which is different from the medical department (to be referred to as a request medical department hereinafter) that has requested the examination. In this case, the doctor of the request medical department needs to closely check the contents of the report and, in some cases, issue a referral letter to another medical department or another hospital and urge the patient to have a consultation in the medical department that can appropriately handle the contents of the report.

However, the appropriate response is sometimes omitted. For example, there is a case in which no appropriate response was made to a diagnostic interpretation report returned from radiology. More specifically. X-ray CT imaging was executed for a patient taken to a hospital by an ambulance, and a doctor on duty in the emergency room takes care of the disease/wound that was the main factor of the emergency transportation. On the other hand, although the diagnostic interpretation report of X-ray CT imaging concerning the patient described a suspicion of lung cancer, the acute care physician failed to make a referral to an appropriate medical department. That is, if another medical department specializes in a disease described in a diagnostic interpretation report, no support is made concerning the disease in some cases. For example, if another medical department specializes in the contents of findings or the contents of impression in the report of an examination result, an action such as issuance of a referral letter to another medical department is sometimes omitted.

### BRIEF DESCRIPTION OF THE DRAWINGS

FIG. 1 is a block diagram showing examples of the arrangement of a consultation support system according to the embodiment and an external apparatus and external systems connected to the consultation support system via a network;

FIG. 2 is a block diagram showing examples of the arrangement of an HIS (Hospital Information System) to which the consultation support system is applied and an external apparatus and external systems connected to the HIS via the network in the embodiment;

FIG. 3 is a view showing an example of a disease correspondence table according to the embodiment;

FIG. 4 is a flowchart showing an example of the procedure of referral letter issuance support processing in the embodiment;

FIG. 5 is a flowchart showing an example of the procedure of referral letter issuance support processing in the embodiment;

FIG. 6 is a view showing an example of confirmation information displayed on a display together with an order input screen in a case in which the number of decided handling medical departments is one in the embodiment;

FIG. 7 is a view showing an example of confirmation information displayed in a case in which a confirmation request was executed from a request medical department to a decided handling medical department in the past in the embodiment;

FIG. 8 is a view showing an example of confirmation information displayed together with an order input screen in a case in which the number of extracted names of diseases is one, and the number of decided handling medical departments is two in the embodiment;

FIG. 9 is a view showing an example of confirmation information displayed together with an order input screen in a case in which there are two decided handling medical departments respectively corresponding to two extracted disease names in the embodiment;

FIG. 10 is a view showing an example of an in-hospital referral letter displayed on the display in the embodiment;

FIG. 11 is a view showing an example of an in-hospital referral letter displayed on the display in response to pressing of a referral letter generation button shown in FIGS. 8 and 9 in the embodiment;

FIG. 12 is a view showing an example of a warning superimposed on an electronic medical record displayed on the display in the embodiment;

FIG. 13 is a view showing an example of a warning superimposed on an electronic medical record displayed on the display in the embodiment;

FIG. 14 is a view showing an example of a warning superimposed on an electronic medical record displayed on the display in the embodiment;

FIG. 15 is a view showing an example of a warning superimposed on an electronic medical record displayed on the display in the embodiment;

FIG. 16 is a view showing an example of a period correspondence table according to the embodiment;

FIG. 17 is a view showing an example of a referral letter suggestion notification superimposed on an electronic medical record displayed on the display in a decided handling medical department in the embodiment;

FIG. 18 is a block diagram showing the arrangement of an RIS (Radiology Information System) including the consultation support system and examples of an external apparatus and external systems connected to the RIS via the network according to the first modification of the embodiment;

FIG. 19 is a view showing an example of a disease correspondence table according to the second modification of the embodiment;

FIG. 20 is a flowchart showing an example of the procedure of referral letter issuance support processing according to the second modification of the embodiment;

FIG. 21 is a flowchart showing an example of the procedure of referral letter issuance support processing according to the third modification of the embodiment;

FIG. 22 is a view showing an example of confirmation information displayed on a display together with an order input screen in the third modification of the embodiment;

FIG. 23 is a view showing an example of confirmation information displayed on a display screen of an electronic medical record concerning an object at the next time of consultation in the third modification of the embodiment; and

FIG. 24 is a flowchart showing an example of the procedure of referral letter issuance support processing according to the fourth modification of the embodiment.

### DETAILED DESCRIPTION

In general, according to one embodiment, a consultation support system includes processing circuitry. The processing circuitry is configured to extract information that specifies or suggests a type of an abnormality in a result of an examination concerning an object from an analysis result obtained by analyzing the result of the examination. The processing circuitry is configured to decide a handling destination capable of making a consultation about the abnormality specified or suggested in the analysis result using the extracted information that specifies or suggests the type of the abnormality. The processing circuitry is configured to display information concerning a referral letter to the decided handling destination on a display if a request source that has requested the examination is not included in the decided handling destination.

It is an object to reduce omissions in the issuance of referral letters for examination results.

A consultation support system according to this embodiment will now be described with reference to the accompanying drawings. Note that the same reference numerals denote constituent elements having almost the same functions and arrangements in the following explanation, and a repetitive description will be made only when necessary. In addition, “consultation support system” may be replaced with another name such as “medical information processing apparatus”.

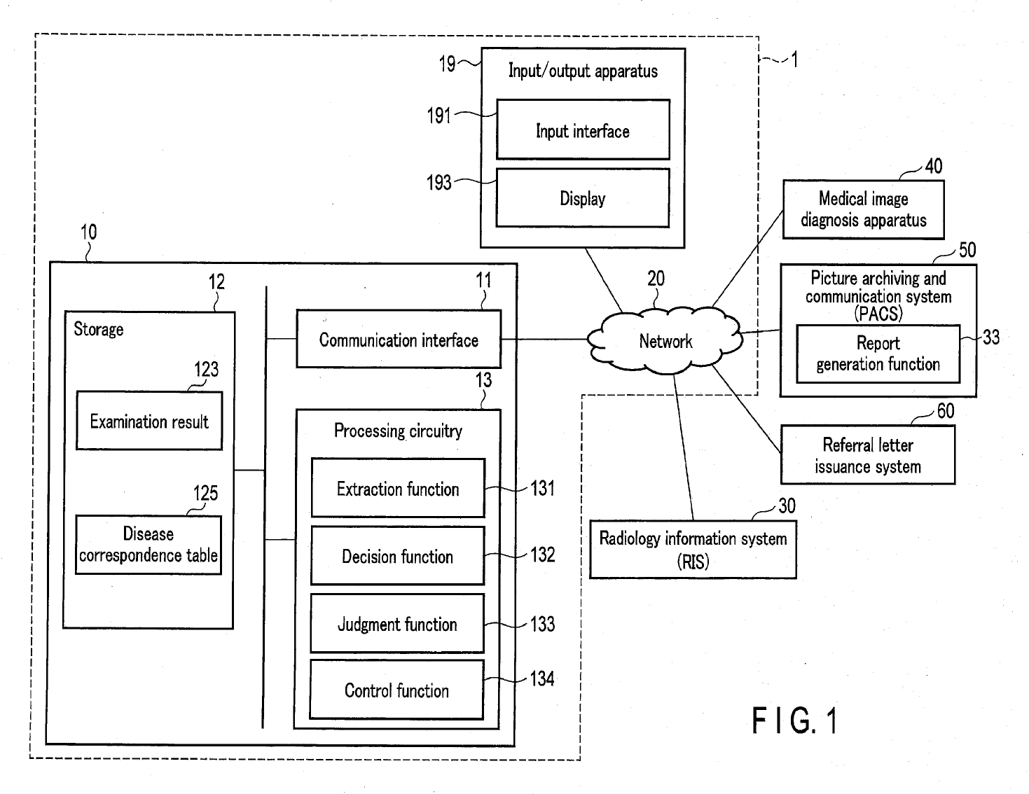

FIG. 1 is a block diagram showing the arrangement of a consultation support system 1 according to this embodiment and examples of an external apparatus and external systems connected to the consultation support system 1 via a network 20. The consultation support system 1 includes a consultation support server 10, and an input/output apparatus 19 connected via the network 20. Constituent elements in the consultation support server 10 will be described later.

The network 20 is, for example, a LAN (Local Area Network). Note that the connection to the network 20 can be either wired connection or wireless connection. Additionally, if security is ensured by a VPN (Virtual Private Network) or the like, the connected network is not limited to the LAN. Connection to a public communication line such as the Internet may be made.

For example, a radiology information system (to be referred to as an RIS hereinafter) 30, a medical image diagnosis apparatus 40, a picture archiving and communication system (to be referred to as a PACS hereinafter) 50, a referral letter issuance system 60, and the like are connected to the consultation support system 1 via the network 20.

The RIS 30 manages information concerning a radiological examination work. The information concerning the radiological examination work includes patient information, examination order information, an exposure record, and the like. Upon receiving patent information and examination order information output from a hospital information system (to be referred to as an HIS hereinafter) server (not shown), the RIS 30 generates, based on the received patient information and examination order information, examination appointment information necessary to operate the medical image diagnosis apparatus 40. The RIS 30 transmits the generated examination appointment information to the medical image diagnosis apparatus 40.

The medical image diagnosis apparatus 40 executes an examination based on the examination appointment information transmitted from the RIS 30. The medical image diagnosis apparatus 40 includes, for example, an X-ray computed tomography imaging apparatus, an X-ray diagnosis apparatus, a magnetic resonance imaging apparatus, a nuclear medicine diagnosis apparatus, an ultrasonic diagnosis apparatus, and the like. The medical image diagnosis apparatus 40 generates a medical image by executing the examination. Examples of the medical images are an X-ray CT image, an x-ray image, an MRI image, a nuclear medicine image, and an ultrasonic image. The medical image diagnosis apparatus 40 converts the generated medical image into a format complying with, for example, the DICOM (Digital Imaging and Communication in Medicine) standard, thereby generating a medical image file. The medical image diagnosis apparatus 40 outputs the generated medical image file to the PACS 50 or the like.

The PACS 50 has a report generation function 30 to be executed by a processor (not shown). The PACS 50 stores the medical image file output from the medical image diagnosis apparatus 40. By the report, generation function 33, the PACS 50 displays a medical image output from the medical image diagnosis apparatus 40 or a medical image stored in the PACS 50 on the display of a report generation apparatus (not shown). The PACS 50 displays, on the display, a graphical user interface (to be referred to as a report generation GUI (Graphical User Interface) hereinafter) configured to input necessary items to a generation screen of a report of image diagnosis, that is, a diagnostic interpretation report. According to an input by an operator, the PACS 50 generates a diagnostic interpretation report using the report generation GUI for the medical image displayed on the display. The PACS 50 outputs the generated diagnostic interpretation report to the HIS in association with the medical image. In addition, the PACS 50 stores the generated diagnostic interpretation report in association with the medical image.

Furthermore, the PACS 50 stores the generated diagnostic interpretation report in association with a medical image referred to by the operator at the time of generation of the diagnostic interpretation report. In accordance with a request from an input/output terminal such as the input/output apparatus 19, the PACS 50 outputs the stored medical image file to the input/output apparatus 19 together with the diagnostic interpretation report.

The referral letter issuance system 60 cooperates with the HIS including an electronic medical record system (not shown). In accordance with an instruction of the operator on the input/output apparatus 19, the referral letter issuance system issues a referral letter (consultation information providing document) generated in the consultation support system 1. The destination of the referral letter can be either the inside of the hospital or the outside of the hospital. Note that the referral letter issuance system 60 may be included in the consultation support server 10.

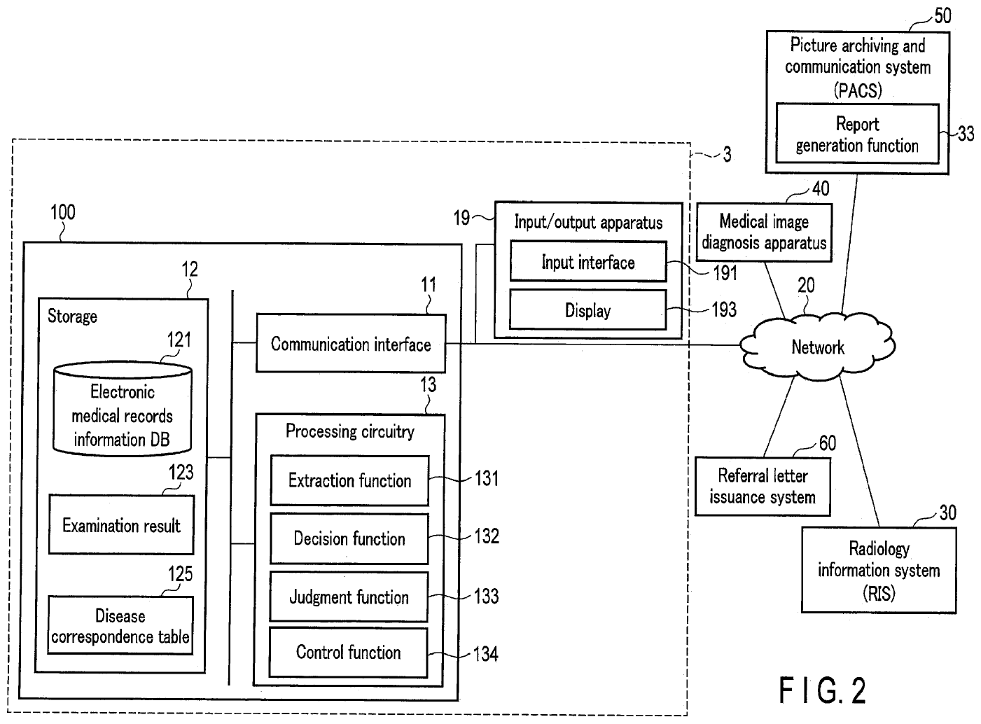

Hereinafter, the consultation support system I is assumed to be included in an HIS for a more detailed description. FIG. 2 is a block diagram showing the arrangement of an HIS 3 to which the consultation support system 1 is applied and examples of an external apparatus and external systems connected to the HIS 3 via the network 20. An HIS server 100 includes constituent elements of the consultation support server 10. The HIS 3 includes the HIS server IOC and the input/output apparatus 19. Note that the input/output apparatus 19 may be connected to a plurality of HIS servers 100. For example, the RIS 30, the medical image diagnosis apparatus 40, the PACS 50, the referral letter issuance system 60, and the like are connected to the HIS 3 via the network 20. Note that the referral letter issuance system 60 may be included in the HIS server 100. In addition, the consultation support system 1 may be included in the RIS 30. A case in which, the consultation support system 1 is included in the RIS 30 will be described in the first modification to be described later.

The HIS server 100 includes a communication interface (communication unit) 11, a storage (storage unit) 12, and processing circuitry (processing unit) 13. The communication interface 11, the storage 12, and the processing circuitry 13 are connected to be communicable with each other via a bus by, for example, wired connection or wireless connection. The storage (storage unit) is an example of a memory.

The communication interface 11 is a network interface that performs transmission control of communications among the input/output apparatus 19, the RIS 30, the medical image diagnosis apparatus 40, the PACS 50, and the referral letter issuance system 60 via the network 20. Under the control of a control function 134 of the processing circuitry 13, the communication interface 11 acquires, via the network 20, a result (to be referred to as an examination result hereinafter) of an examination concerning an object. The communication interface 11 outputs the acquired examination result to the storage 12 and the processing circuitry 13. The examination result corresponds to, for example, a result of a specimen examination for an object or an examination report (image diagnosis report) such as a diagnostic interpretation report.

Hereinafter, the examination result is assumed to be a diagnostic interpretation report for a more detailed description. The diagnostic interpretation report includes, for example, findings and impression for a medical image generated by the medical image diagnosis apparatus 40. The diagnostic interpretation report is generated by, for example, a radiologist based on the medical image using the report generation function 33 of the PACS 50. Note that the report generation function 33 may be included in the processing circuitry 13 or the like, or may be separately connected to the network 20 as a report generation apparatus independent of the PACS 50.

Note that the communication interface 11 may acquire an analysis result for the diagnostic interpretation report generated by the report generation function 33. The analysis result is a result obtained by analyzing a natural sentence in the examination result such that the name of a disease in the examination result can be extracted. That is, the analysis result is a result of natural language processing for the diagnostic interpretation report, which is, for example, a result (structured report) of structured analysis of extracting phrases in natural sentences described in the diagnostic interpretation report and classifying the phrases on an attribute basis or a result of morphological analysis for natural sentences described in the diagnostic interpretation report. At this time, the communication interface 11 outputs the analysis result obtained by analyzing the diagnostic interpretation report by the report generation function 33 or the like to the processing circuitry 13.

The storage 12 is implemented by, for example, a RAM (Random Access Memory) that stores various kinds of information, a semiconductor memory element such as a flash memory, a hard disk drive, a solid state drive, an optical disk, or the like. Note that the storage 12 may further include a nonvolatile memory such as a ROM (Read Only Memory). In addition, the storage 12 may be a drive device configured to read/write various kinds of information from/to a portable storage medium such as a CD (Compact Disc)-ROM drive, a DVD drive, or a flash memory.

The storage 12 stores control programs such as an operating system in the consultation support system 1, various kinds of application programs, and display data of various kinds of operation screens (GUIs) attached to these programs. The storage 12 stores programs corresponding to various kinds of functions to be executed by the processing circuitry 13. In addition, the storage 12 stores an electronic medical records information DB (Data Base) 121. Electronic medical record information is stored in the electronic medical records information DB 121 on a patient basis. The electronic medical record information includes chief complaint information, finding information, diagnosis information, and treatment plan information in each consultation date and order information in each consultation date. The electronic medical record information also includes a diagnosed disease name, an examination result, an examination result including a diagnostic interpretation report, anamnesis, and the like.

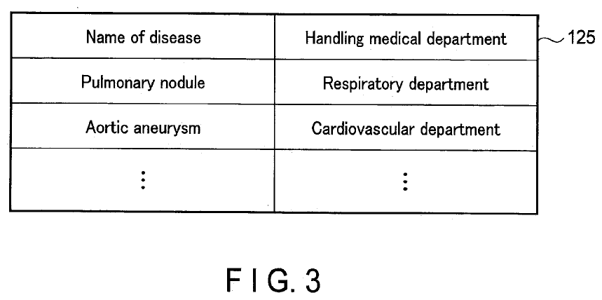

The storage 12 stores an examination result 123 acquired via the communication interface 11. More specifically, the storage 12 stores, as the examination result 123, a diagnostic interpretation report for a medical image of an object acquired by the medical image diagnosis apparatus 40. In the examination result including the diagnostic interpretation report, a medical department (to be referred to as a request medical department hereinafter) that requested the examination is described. The storage 12 stores a disease correspondence table 125 that associates a plurality of medical departments (to be referred to as handling medical departments hereinafter) capable of making a consultation concerning a plurality of diseases with the plurality of diseases. FIG. 3 is a view showing an example of the disease correspondence table 125. As shown in FIG. 3, the disease correspondence table 125 corresponds to a list that associates a plurality of disease names with a plurality of handling medical departments. In the disease correspondence table 125 shown in FIG. 3, the plurality of disease names and the handling medical departments need not always be associated in a one-to-one correspondence, and a plurality of handling medical departments may be associated with one disease name.

The processing circuitry 13 functions as the center of the HIS server 100. The processing circuitry 13 includes, as hardware resources, a processor and memories such as a ROM and a RAM (none are shown). The processing circuitry 13 includes an extraction function 131, a decision function 132, a judgment function 133, and the control function 134. By the control function 134, the processing circuitry 13 integrally controls various kinds of circuitry and functions including the HIS server 100. The various kinds of functions performed by the extraction function 131, the decision function 132, the judgment function 133, and the control function 134 are stored in the storage 12 as a form of programs executable by a computer. The processing circuitry 13 is a processor that reads the programs corresponding to the various kinds of functions from the storage 12 and executes them, thereby implementing the functions corresponding to the programs. In other words, the processing circuitry 13 that has read the programs has the functions in the processing circuitry 13 shown in FIGS. 1 and 2, The extraction function 131, the decision function 132, the judgment function 133, and the control function 134 provided in the processing circuitry 13 are examples of an extraction unit, a decision unit, a judgment unit, and a control unit, respectively. The extraction function 131, the decision function 132, the judgment function 133, and the control function 134 provided in the processing circuitry 13 may be provided as separate circuitry, that is, extraction circuitry, decision circuitry, judgment circuitry, and control circuitry, respectively.

The word “processor” used in the above description means, for example, circuitry such as a CPU (Central Processing Unit), an MPU (Micro Processing Unit), a GPU (Graphical Processing Unit), an ASIC (Application Specific Integrated Circuit), or a programmable logic device (for example, an SPLD (Simple Programmable Logic Device), a CPLD (Complex Programmable Logic Device), or an FPGA (Field Programmable Gate Array)).

Note that in this embodiment, a case in which the extraction function 131, the decision function 132, the judgment function 133, and the control function 134 are implemented by a single processor will be described. However, the present embodiment is not limited to this. For example, control circuitry may be formed by combing a plurality of independent processors, and the processors may implement the extraction function 131, the decision function 132, the judgment function 133, and the control function 134 by executing operation programs.

Processes concerning the extraction function 131, the decision function 132, the judgment function 133, and the control function .134 executed by the processing circuitry 13 will be described in the procedure of referral letter issuance support processing to be described later. Referral letter issuance support processing is processing of executing support of issuance of a referral letter based on an examination result including a diagnostic interpretation report.

The input/output apparatus 19 includes, for example, an input interface (input unit) 191 and a display (display unit) 193. Note that the input/output apparatus 19 may be connected to the network 20.

The input interface 191 accepts various kinds of input operations from the operator, converts each accepted input operation into an electrical signal, and outputs the electrical signal to the processing circuitry 13 via the communication interface 11 and the network 20 in some cases. As the input interface 191, for example, a mouse, a keyboard, a track ball, a switch, a button, a joystick, a touch pad, a touch panel display, and the like can appropriately be used. Note that in this embodiment, the input interface 191 is not limited to interfaces including a physical operation component such as a mouse, a keyboard, a track ball, a switch, a button, a joystick, a touch pad, and a touch panel display. For example, electrical signal processing circuitry configured to receive an electrical signal corresponding to an input operation from an external input device provided independently of the apparatus and output the electrical signal to the processing circuitry 13 is also included in the example of the input interface 191.

Note that the input device connected to the input interface 191 may be an input device provided in another computer connected via the network or the like. For example, the input interface 191 inputs an access to the HIS server 100 based on an instruction of the operator. The input interface 191 inputs contents concerning a consultation to an electronic medical record displayed on the display 193. In addition, the input interface 191 inputs the presence/absence of issuance of a referral letter for an object and the contents of the referral letter.

The display 193 displays various kinds of information under the control of the control function 134 of the processing circuitry 13. The display 193 displays, for example, an electronic medical record, an order input screen, and a referral letter generation screen. As the display 193, various arbitrary displays can appropriately be used. For example, as the display 193, a liquid crystal display, a CRT (Cathode Ray Tube) display, an organic EL (Electro-Luminescence) display, or a plasma display can be used. In addition, the display 193 may be of a desktop type, or may be formed by a tablet terminal or the like capable of wirelessly communicating with the HIS server 100.

The overall arrangement of the HIS 3 including the consultation support system 1 according to this embodiment, has been described above. Processing contents executed by the extraction function 131, the decision function 132, the judgment function 133, and the control function 134 according to this embodiment will be described below together with the procedure of referral letter issuance support processing.

(Referral Letter Issuance Support Processing)

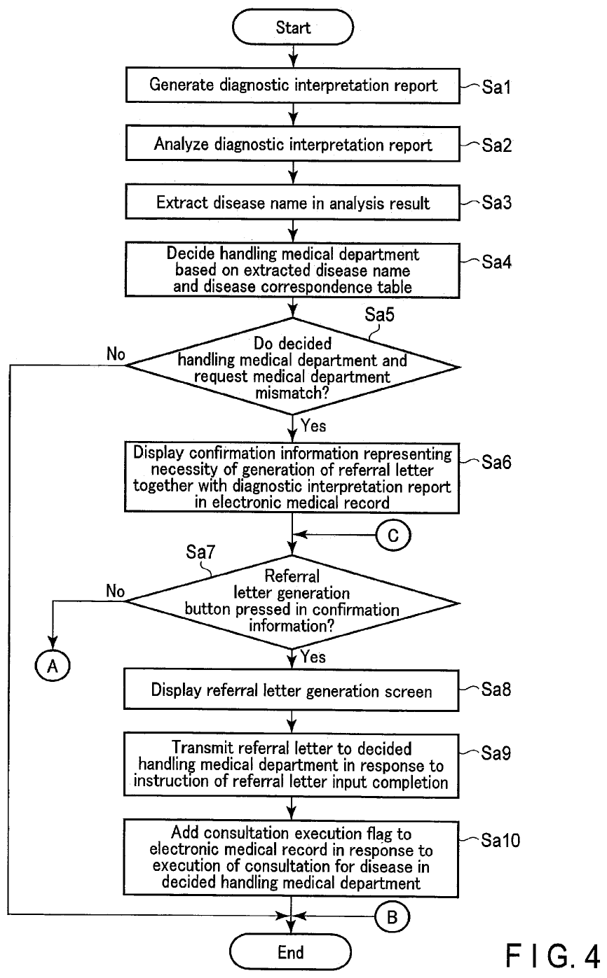

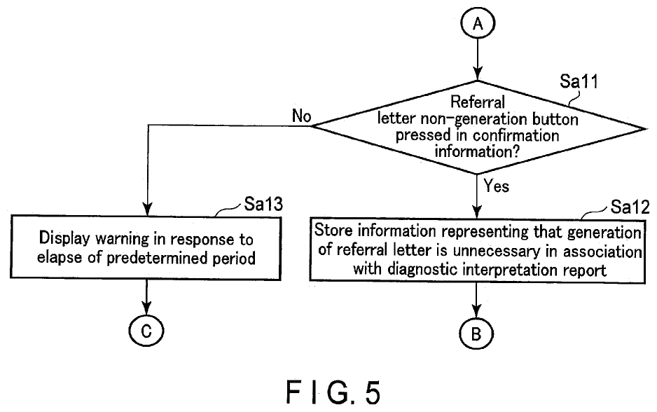

FIGS. 4 and 5 are flowcharts showing an example of the procedure of referral letter issuance support processing.

(Step Sa1)

A diagnostic interpretation report is generated in the PACS 50. The generated diagnostic interpretation report is output to the processing circuitry 13 via the communication interface 11 and the network 20.

(Step Sa2)

By the extraction function 131, the processing circuitry 13 executes natural language processing for the diagnostic interpretation report, and analyzes the diagnostic interpretation report. That is, the processing circuitry 13 analyzes natural sentences in the examination result, thereby acquiring an analysis result that enables extraction of the name of a disease. In other words, the processing circuitry 13 analyzes whether the examination result includes the name of a disease, thereby acquiring an analysis result that enables extraction of the name of a disease. More specifically, the processing circuitry 13 executes, for example, structured analysis or morphological analysis for the diagnostic interpretation report. A program configured to execute structured analysis or morphological analysis is stored in the storage 12 in advance. Note that the natural language processing for the diagnostic interpretation report is not limited to structured analysis and morphological analysis, and another analysis processing may be executed. The processing circuitry 13 generates an analysis result obtained by analyzing the diagnostic interpretation report. Hereinafter, the analysis result is assumed to be a structured report for a more detailed description. Note that if the structured report corresponding to the diagnostic interpretation report is generated by the report generation function 33 and the like in the PACS 50, the processing of this step is processing of reading the structured report from the PACS 50 by the communication interface 11 and outputting the read structured report to the processing circuitry 13.

(Step Sa3)

The processing circuitry 13 extracts the name of a disease (to be referred to as a disease name hereinafter) in the analysis result by the extraction function 131. More specifically, the processing circuitry 13 extracts the disease name in the examination result, based on the analysis result corresponding to the examination result concerning the object. At this time, a plurality of disease names may be extracted from the analysis result. For example, the processing circuitry 13 executes matching processing between a plurality of words in the structured report and, for example, the names of diseases stored in the memory of its own or the like. The processing circuitry 13 thus extracts a disease name described in the impression of the diagnostic interpretation report. Note that the processing circuitry 13 may extract a disease name described in the findings of the diagnostic interpretation report. In addition, the processing circuitry 13 may extract the name of a request medical department in the analysis result.

Note that the processing circuitry 13 may limit the range to apply the matching processing for extracting a disease name to the structured report corresponding to the impression. At this time, since a disease name described in the findings is not extracted, overextraction of disease names can be avoided.

(Step Sa4)

The processing circuitry 13 decides a handling medical department by the decision function 132 based on the extracted disease name and the disease correspondence table. That is, the processing circuitry 13 decides a handling medical department, capable of having a consultation about the extracted disease using the extracted disease name and the disease correspondence table. The disease name described in the impression of the diagnostic interpretation report is thus specified. If a plurality of disease names are extracted from the analysis result, the processing circuitry 13 decides a handling medical department for each of the plurality of extracted disease names. Note that if a plurality of handling medical departments are associated with each of the extracted disease names in the disease correspondence table, the processing circuitry 13 decides a plurality of handling medical departments capable of having a consultation about the extracted diseases using the extracted disease name and the disease correspondence table.

(Step Sa5)

The processing circuitry 13 judges, by the judgment function 133, whether the decided handling medical department (to be referred to as a decided handling medical department hereinafter) matches the request medical department. More specifically, the processing circuitry 13 compares the request medical department that has requested the examination with the decided handling medical department, thereby judging matching or mismatching between the request medical department and the decided handling medical department. If the request medical department and the decided handling medical department do not match (YES in step Sa5), processing of step Sa6 is executed. If the request medical department and the decided handling medical department match (NO in step Sa5), the processing circuitry 13 ends the referral, letter issuance support processing.

If a plurality of decided handling medical departments exist for one extracted disease name, the processing circuitry 13 judges whether the request medical department is included in the plurality of decided handling medical departments. More specifically, the processing circuitry 13 judges, for each of the plurality of decided handling medical departments, whether the decided handling medical department matches the request medical department, thereby judging whether there is a decided handling medical department that matches the request medical department. If the request medical department is not included in the plurality of decided handling medical departments (YES in step Sa5), that is, if the decided handling medical department and the request medical department do not match concerning all of the plurality of decided handling medical departments, processing of step Sa6 is executed. If the request medical department is included in the plurality of decided handling medical departments (NO in step Sa5), that is, if at least one decided handling medical department that matches the request medical department exists among the plurality of decided handling medical departments, the processing circuitry 13 ends the referral letter issuance support processing.

Note that if one decided handling medical department exists for each of the plurality of disease names extracted from the analysis result, the processing circuitry 13 judges whether at least one of the plurality of decided handling medical departments matches the request medical department. More specifically, the processing circuitry 13 judges, for each of the plurality of decided handling medical departments, whether the decided handling medical department matches the request medical department, and judges whether there is a decided handling medical department that does not match the request medical department. If at least one of the plurality of decided handling medical departments does not match the request medical department (YES in step Sa5), that is, at least one decided handling medical department that does not match the request, medical department exists among the plurality of decided handling medical departments, processing of step Sa6 is executed. If the plurality of decided handling medical departments match the request medical department (NO in step Sa5), that is, if the decided handling medical department and the request medical department match concerning all of the plurality of decided handling medical departments, the processing circuitry 13 ends the referral letter issuance support processing.

(Step Sa6)

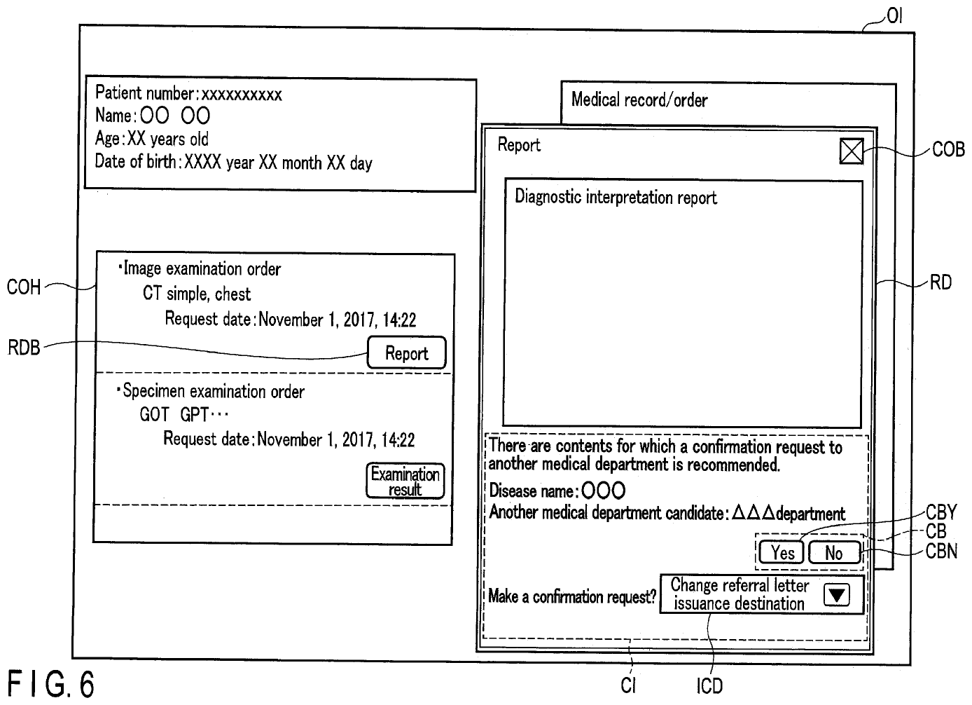

By the control function 134, the processing circuitry 13 displays, on the display 193, confirmation information representing the necessity of generation of a referral letter from the request medical department to the decided handling medical department together with the diagnostic interpretation report in the electronic medical record. FIG. 6 is a view showing an example of confirmation information CI displayed on the display 193 together with an order input screen OI in this step in a case in which the number of decided handling medical departments is one. As shown in FIG. 6, when the operator presses a report display button RDB in a medical record/order history display region COH of the order input screen OI, the processing circuitry 13 displays the confirmation information CI on the display 193 together with a diagnostic interpretation report RD.

The confirmation information CI includes, for example, character information “There are contents for which a confirmation request to another medical department is recommended. Disease, name: ◯◯◯, Another medical department candidate: ΔΔΔ department. Make a confirmation request?”, a button CB used to decide the necessity of a confirmation request, and a button (to be referred to as an issuance destination change button hereinafter) ICD capable of changing the issuance destination of a referral letter. The disease name displayed in the confirmation information CI is the name of the disease extracted by the processing of step Sa3. In addition, the other medical department candidate displayed in the confirmation information CI is the decided handling medical department decided by the processing of step Sa3. Note that the display of the button ICD capable of changing the issuance destination of a referral letter may be omitted.

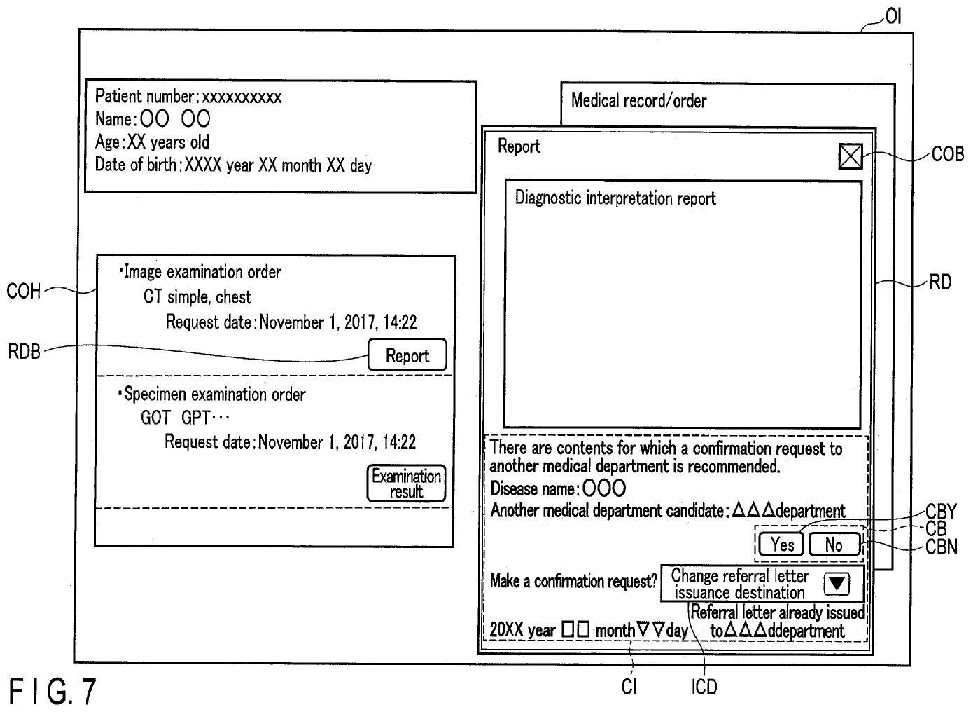

Note that the words and the like in the confirmation information CI shown in FIG. 6 are merely examples, and are not limited to the description contents shown in FIG. 6. For example, if a confirmation request was executed from the request medical department to the decided handling medical department in the past, by the control function 134, the processing circuitry 13 displays, on the display 193, the confirmation information CI additionally including the date of the issuance of the referral letter to the decided handling medical department and character information representing that the referral letter was already issued. FIG. 7 is a view showing an example of the confirmation information CI displayed in a case in which a confirmation request was executed from the request medical department to the decided handling medical department in the past. At this time, the operator can confirm that the referral letter was already issued to the other medical department candidate ΔΔΔ department.

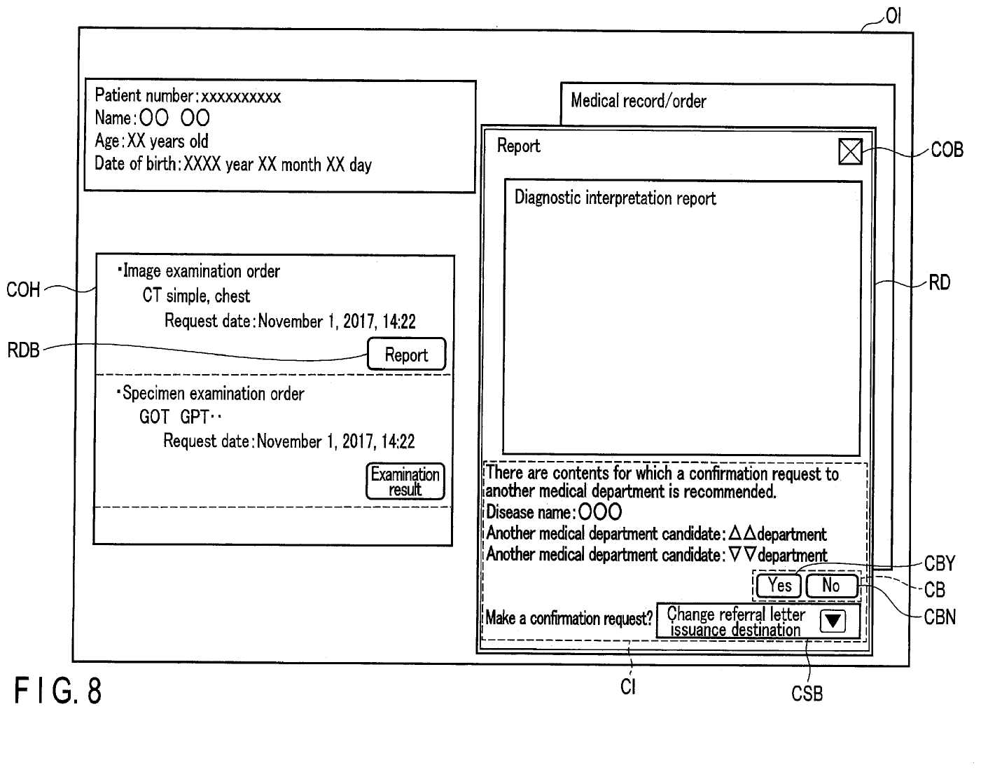

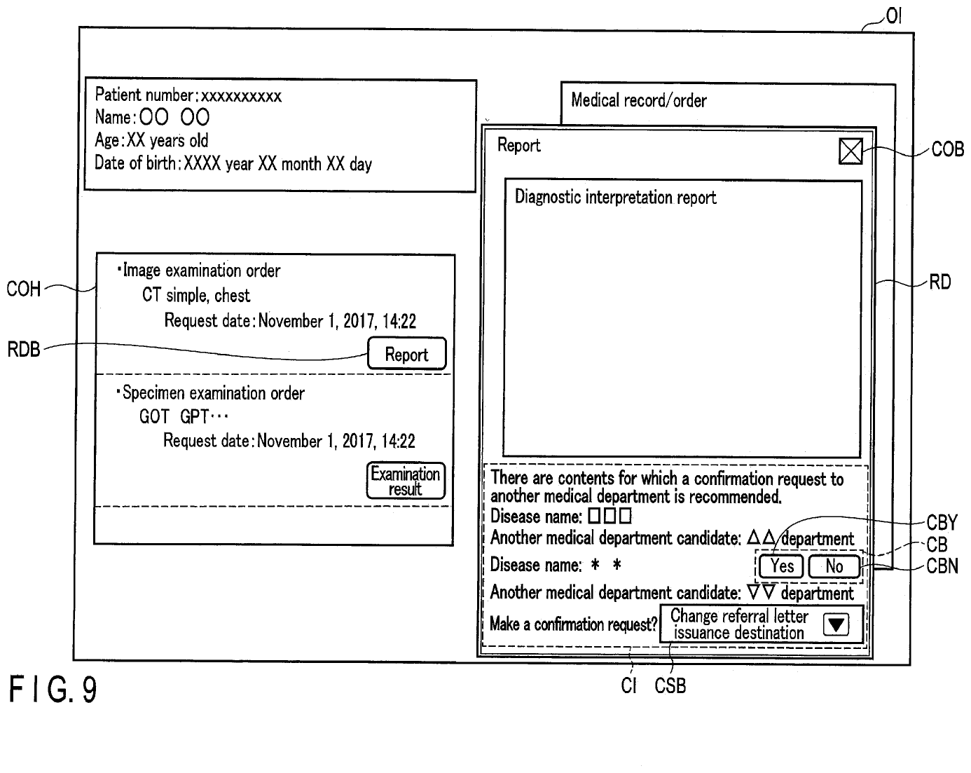

FIG. 8 is a view showing an example of the confirmation information CI displayed together with the order input screen OI in a case in which the number of extracted names of diseases is one, and the number of decided handling medical departments associated with the extracted disease name is two. FIG. 9 is a view showing an example of the confirmation information CI displayed together with the order input screen OI in a case in which there are two decided handling medical departments respectively corresponding to two extracted disease names. As shown in FIGS. 8 and 9, the confirmation information CI includes character information, the button CB used to decide the necessity of a confirmation request, and a button (to be referred to as an issuance destination selection button hereinafter) CSB capable of selecting the issuance destination of a referral letter.

Note that the confirmation information CI shown in FIGS. 6, 7, 8, and 9 is displayed when contents whose confirmation should be requested to another medical department different from the request medical department are included in the examination result including the diagnostic interpretation report. That is, if the request medical, department and the decided handling medical department match (NO in step Sa5), the confirmation information CI in the diagnostic interpretation report RD is not displayed on the display 193. In addition, if there are a plurality of decided handling medical departments, and the request medical department matches all of the decided handling medical departments (NO in step Sa5), the confirmation information CI in the diagnostic interpretation report RD is not displayed on the display 193.

(Step Sa7)

When a referral letter generation button CBY is pressed based on an instruction of the operator in the confirmation information CI displayed on the display 193 (YES in step Sa7), the processing circuitry 13 executes processing of step Sa8 by the control function 134. Note that if the issuance destination change button ICD or the issuance destination selection button CSB is pressed in place of the referral letter generation button CBY, the processing circuitry 13 displays the names of a plurality of other medical departments on the display 193 as, for example, a pull-down. If one of the plurality of displayed other medical departments is selected based on an instruction of the operator, the processing circuitry 13 executes processing of step Sa8 concerning the selected another medical department.

If the referral letter generation button CBY is not pressed based on an instruction of the operator in the confirmation information CI displayed on the display 193 (NO in step Sa7), the processing circuitry 13 executes processing of step Sa11 by the control function 134.

(Step Sa8)

The processing circuitry 13 displays a referral letter generation screen (GUI) on the display 193 by the control function 134. That is, if the operator selects that generation of a referral letter is necessary for the confirmation information CI by pressing the referral letter generation button CBY, the issuance destination change button ICD, or the issuance destination selection button CSB in the confirmation information CI (YES in step Sa7), the processing circuitry 13 associates the selection result representing that generation of a referral letter is necessary with the examination result, and displays, on the display 193, an input screen that enables input of a plurality of items in the referral letter.

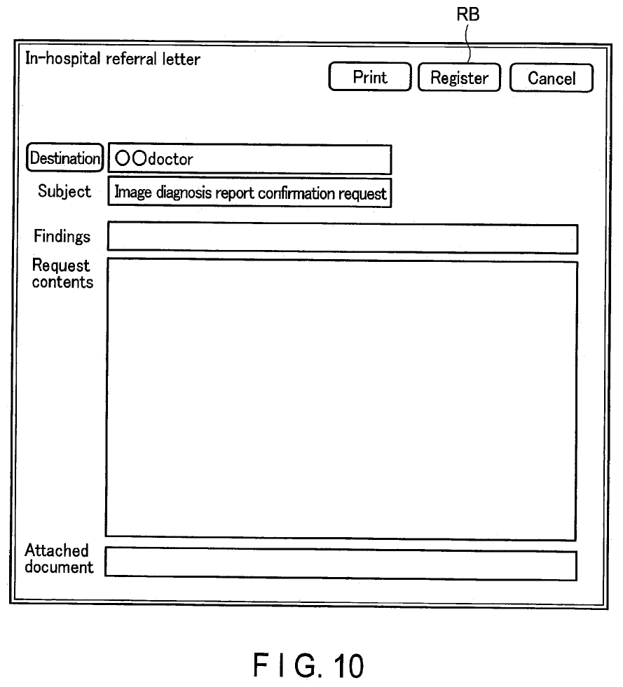

FIG. 10 is a view showing an example of an in-hospital referral letter displayed on the display 193 in this step in response to the pressing of the referral letter generation button CBY in FIGS. 6 and 7, selection of another medical department by the pressing of the issuance destination change button ICD in FIGS. 6 and 7, or selection of another medical department by the pressing of the issuance destination change button ICD in FIGS. 8 and 9. Note that in the referral letter shown in FIG. 10, the issuance destination change button ICD may further be displayed.

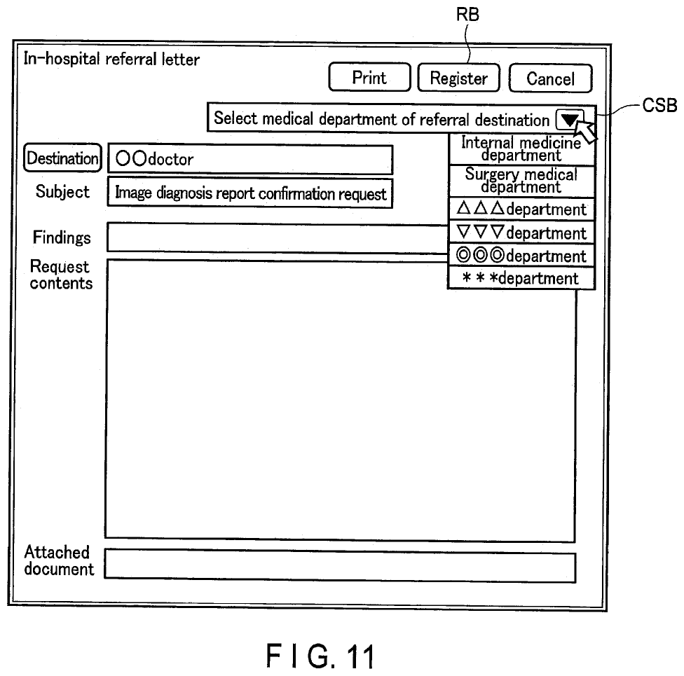

FIG. 11 is a view showing an example of an in-hospital referral letter displayed on the display 193 in this step in response to the pressing of the referral letter generation button CBY in FIGS. 8 and 9. As the difference between FIGS. 10 and 11, the issuance destination selection button CSB is displayed in FIG. 11 because the issuance destination of the referral letter is a plurality of medical departments. At this time, a button used by the operator to select the medical department of the referral destination is provided in the in-hospital referral letter. As shown in FIG. 11, if the issuance destination selection button CSB is pressed in the in-hospital referral letter, for example, a list of medical departments of referral destinations is displayed as a pull-down.

Note that if the operator selects that generation of a referral letter is necessary for the confirmation information CI (YES in step Sa7), the processing circuitry 13 may generate words concerning the disease and the decided handling medical department by the control function 134 based on the analysis contents of the examination result and then display, on the display 193, a referral letter input screen with the generated words in at least one item of the plurality of items in the referral letter. Examples of generated words are the destination of the referral letter, subject, findings, and impression in the referral letters shown in FIGS. 10 and 11.

(Step Sa9)

In response to an instruction of referral letter input completion, the processing circuitry 13 transmits the generated referral letter to the decided handling medical department via the referral letter issuance system 60 by the control function 134. More specifically, in response to the pressing of a registration button RB in the in-hospital referral letter shown in FIG. 10 or 11, the processing circuitry 13 transmits the in-hospital referral letter to the decided handling medical department. Note that even if the referral destination is an affiliated medical facility outside the hospital, the processing circuitry 13 transmits the referral letter to the decided handling medical department in the affiliated consultation facility in response to the pressing of the registration button RB. Note that if the referral destination is a non-affiliated medical facility outside the hospital, the processing circuitry 13 issues the referral letter via the referral letter issuance system 60 in response to the pressing of the registration button RB.

(Step Sa10)

In response to execution of a consultation about the disease in the decided handling medical department, the processing circuitry 13 adds a consultation execution flag to the electronic medical record by the control function 134. More specifically, if the operator selects that generation of a referral letter is necessary for the confirmation information (YES in step Sa7), the referral letter generated based on the instruction of the operator is output to the decided handling medical department, and a consultation concerning the disease in the examination result is executed for the object in the decided handling medical department, the processing circuitry 13 sets, in the electronic medical record concerning the object, a flag regarding execution of a consultation about the extracted disease. When the processing of this step is completed, the processing circuitry 13 ends the referral letter issuance support processing.

(Step Sa11)

If a referral letter non-generation button CBN is pressed based on an instruction of the operator in the confirmation information CI displayed on the display 193 (YES in step Sa11), the processing circuitry 13 executes processing of step Sa12 by the control function 134. If the referral letter non-generation button CBN is not pressed based on an instruction of the operator in the confirmation information CI displayed on the display 193 (NO in step Sa11), the processing circuitry 13 executes processing of step Sa13.

(Step Sa12)

The processing circuitry 13 stores information (to be referred to as referral letter unnecessity information hereinafter) representing that generation of a referral letter is unnecessary in the storage 12 in association with the diagnostic interpretation report by the control function 134. Note that the referral letter unnecessity information may be associated with the electronic medical record of the object. More specifically, if the operator selects that generation of a referral letter is unnecessary for the confirmation information CI (YES in step Sa11), the processing circuitry 13 associates the selection result representing that generation of a referral letter is unnecessary with the examination result. At this time, as the referral letter unnecessity information, the processing circuitry 13 stores, for example, the name of the doctor in the request medical department, who has selected that generation of a referral letter is unnecessary, and the date/time of selection of the unnecessity of generation of a referral letter in the storage 12 in association with the diagnostic interpretation report and the electronic medical record. When the processing of this step is completed, the processing circuitry 13 ends the referral letter issuance support processing.

(Step Sa13)

The processing circuitry 13 displays a warning on the display 193 An the request medical department in response to the elapse of a predetermined period by the control function 134. The predetermined period is a period set in default, for example, several hours, several days, several weeks, or the like and is stored in the storage 12. More specifically, in a case in which selection in the confirmation information CI is not input for a predetermined period after a close button COB in the diagnostic interpretation report is pressed, or after the display of the order input screen OI is forcibly terminated, the processing circuitry 13 displays a warning in the display screen of the electronic medical record when the electronic medical record concerning the object is reaccessed. Note that in the case in which selection in the confirmation information CI is not input for a predetermined period, the processing circuitry 13 may display the warning on the display of a terminal accessed by at least one of the doctor in charge of the object and a doctor belonging to the medical department to which the doctor in charge belongs. After the processing of this step, processing from step Sa7 is repeated.

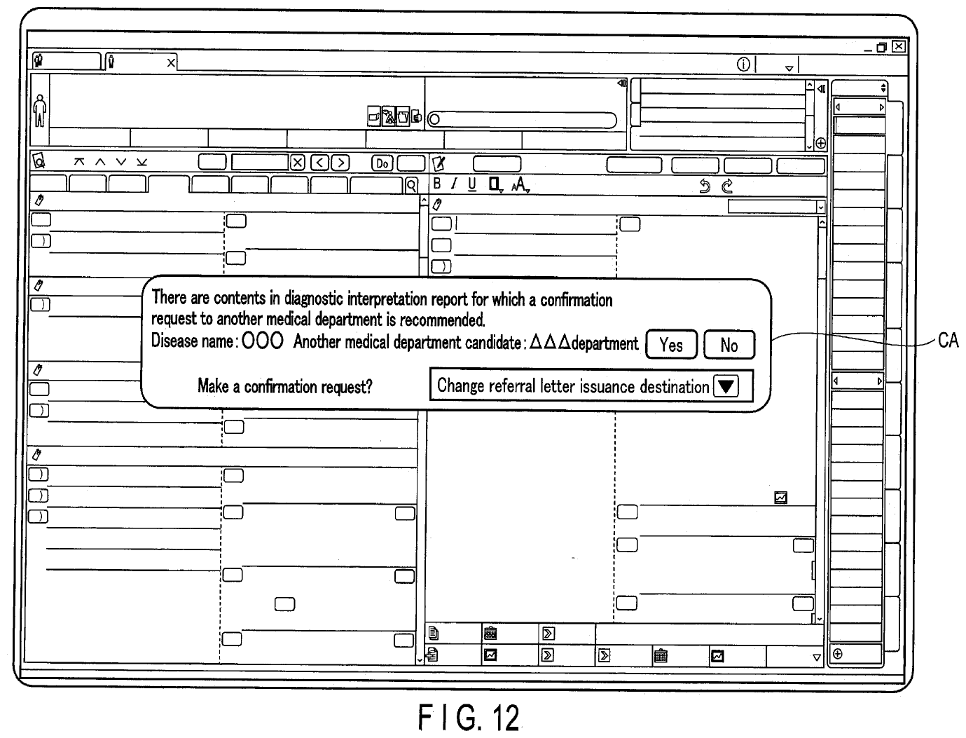

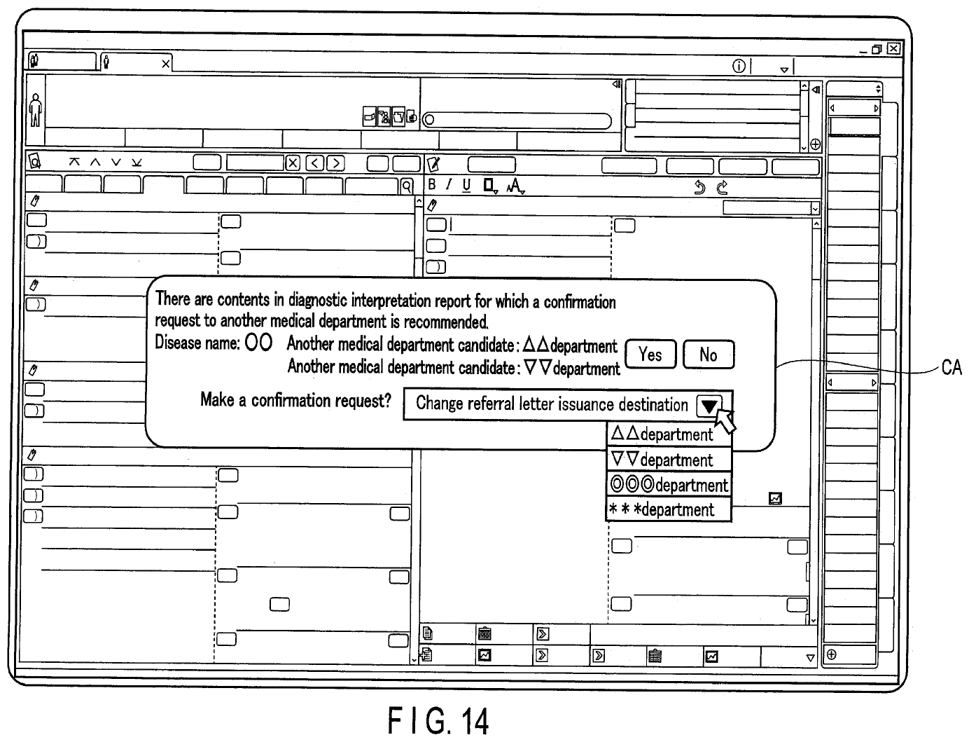

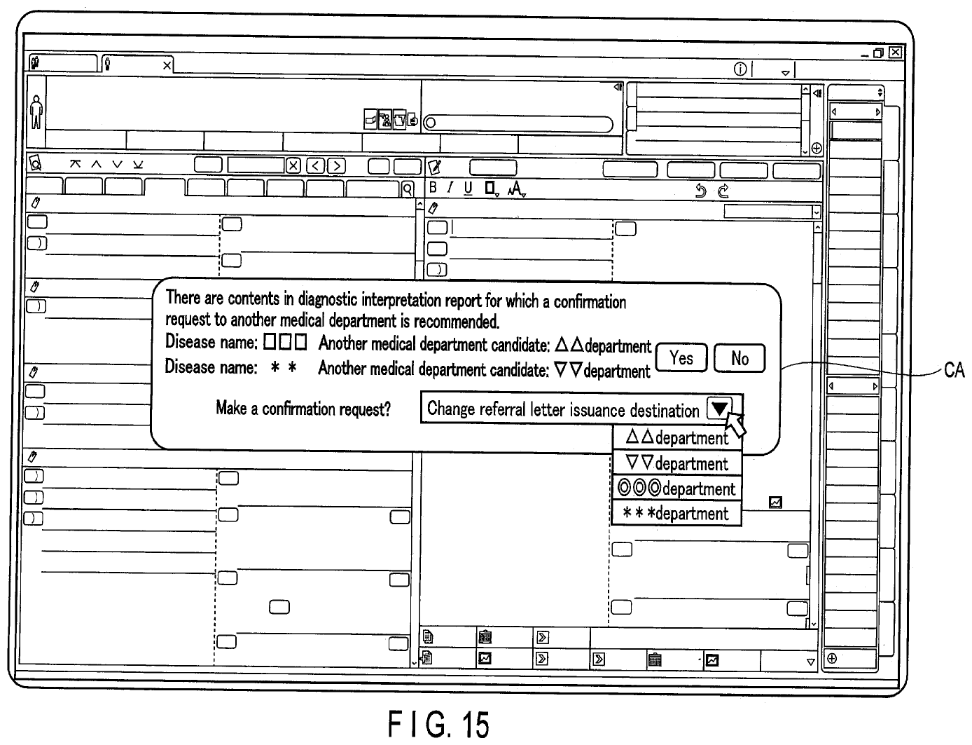

FIGS. 12, 13, 14, and 15 are views showing examples of warnings superimposed on an electronic medical record displayed on the display 193. warnings CA shown in FIGS. 12, 13, 14, and 15 correspond to the pieces of confirmation information CI shown in FIGS. 5, 7, 8, and 9, respectively, although the words are different from the. words of the pieces of confirmation information CI shown in FIGS. 6, 7, 8, and 9. As shown in FIGS. 12, 13, 14, and 15, the warning CA is displayed on the display 193 together with the disease name and another medical department candidate in a display form that enables selection of a referral letter issuance destination based on an instruction of the operator. Note that the -warning CA displayed on the display 193 is not limited to the described contents of the confirmation information CI and may be displayed in any display form as long as it enables selection of the presence/absence of generation of a referral letter.

Note that a predetermined period may be associated with each of a plurality of diseases in accordance with the degree of exacerbation of the disease, that is, the degree of progress of deterioration of the condition. At this time, the storage 12 stores a period correspondence table that associates a plurality of predetermined periods with a plurality of diseases in accordance with the degree of exacerbation of each disease. Note that the storage 12 may store the period correspondence table incorporated in the disease correspondence table 125.

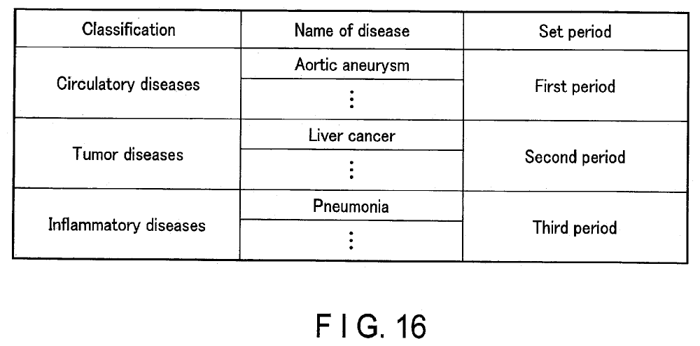

FIG. 16 is a view showing an example of the period correspondence table. As shown in FIG. 16, the names of a plurality of diseases are classified in accordance with the degree of exacerbation. The first period shown in FIG. 16 is shorter than the second period, and is, for example, several days or several hours. The names of diseases that need a quick treatment, for example, circulatory diseases are associated with the first period. The second period shown in FIG. 16 is shorter than the third period, and is, for example, several weeks. The names of diseases that need a treatment at a relatively early stage, for example, tumor diseases are associated with the second period. The third period shewn in FIG. 16 is longer than the second period, and is, for example, several months. The names of diseases that need a follow-up, for example, inflammatory diseases are associated with the third period. Note that the number of periods corresponding to diseases shown in FIG. 16 is not limited to three, and may be two or four or more in accordance with the exacerbations of the diseases.

If the period correspondence table is stored in the storage 12, the processing circuitry 13 decides the predetermined period corresponding to the extracted disease name based on the extracted disease name and the period correspondence table by the control function 134. If selection in the confirmation information CI is not input (NO in step Sa11), the processing circuitry 13 executes display of the warning CA at the interval of predetermined period.

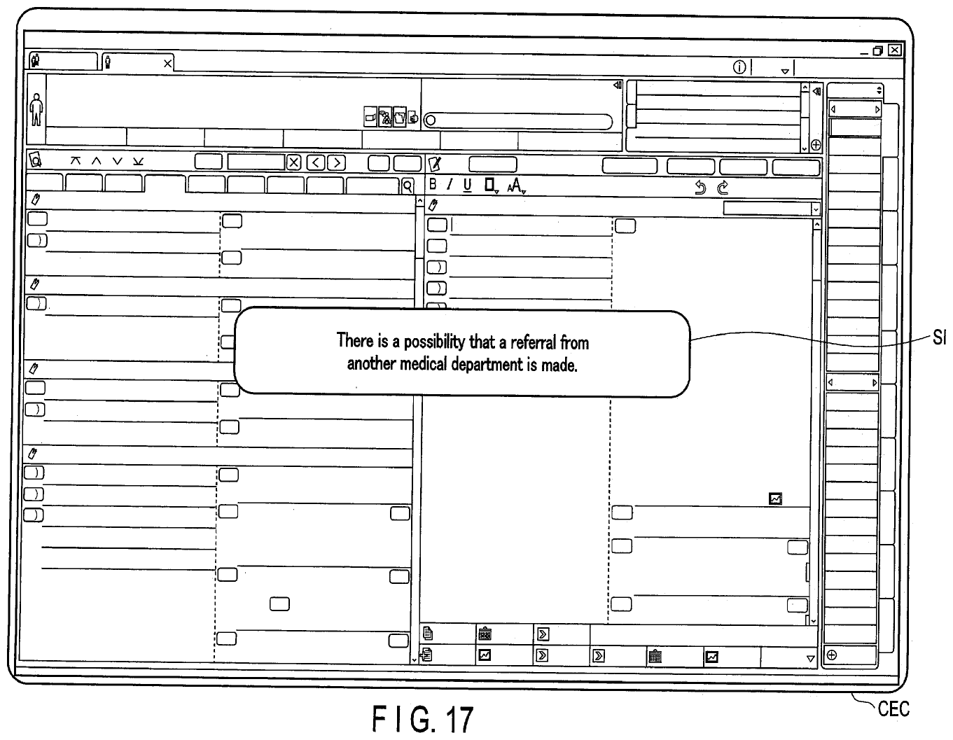

Note that in a case in which selection in the confirmation information CI is not input, and the doctor belonging to the decided handling medical department accesses the electronic medical record of the object, by the control function 134, the processing circuitry 13 may display, on the display screen of the electronic medical record concerning the object, a notification (to be referred to as a referral letter suggestion notification hereinafter) representing that there is a possibility that a referral concerning the object is made to the decided handling medical department. FIG. 17 is a view showing an example of a referral letter suggestion notification SI superimposed on an electronic medical record CEC displayed on the display 193 in the decided handling medical department. When the referral letter suggestion notification SI is displayed, the doctor belonging to the decided handling medical department can grasp the possibility that a referral letter is transmitted from the request medical department. Note that the display of the referral letter suggestion notification SI on the display 193 in the decided handling medical department may be executed during the period from the processing of step Sa6 to the completion of the processing of step Sa9.

According to the above-described arrangement and operation, the following effects can be obtained.

According to the consultation support system 1 of this embodiment, the disease correspondence table 125 that associates a plurality of handling medical departments capable of making a consultation concerning a plurality of diseases with the names of the plurality of diseases is stored, the name of a disease in an examination result is extracted from an analysis result obtained by analyzing the result of an examination concerning the object, a handling medical department capable of making a consultation about the extracted medical disease is decided using the extracted disease name and the disease correspondence table, and the request medical department that has requested the examination is compared with the decided handling medical department, thereby judging matching or mismatching between the request medical department and the decided handling medical department. If the request medical department and the decided handling medical department do not match, the confirmation information CI representing the necessity of generation of a referral letter from the request medical department to the decided handling medical department can be displayed on the display 193. Additionally, according to the consultation support system 1, a natural sentence in the examination result is analyzed, thereby acquiring an analysis result that enables extraction of the name of a disease.

That is, according to the consultation support system 1 of this embodiment, information that specifies or suggests the type of an abnormality in the examination result is extracted from the analysis result obtained by analyzing the result of the examination concerning the object, and a handling destination capable of making a consultation about the abnormality specified in the analysis result is decided using the extracted information that specifies the type of the abnormality. If the request source that has requested the examination is not included in the decided handling destination, information about a referral letter to the decided handling destination can be displayed on the display 193.

Note that the request medical department according to this embodiment is an example of the request source. The request source may be, for example, a hospital that has requested an examination. The handling medical department according to this embodiment is an example of the above-described handling destination. The handling destination may be, for example, a medical department provided in the hospital to which the request medical department belongs, may be a medical department belonging to a hospital different from the hospital to which the request medical department belongs, or may be a clinic or the like different from the hospital to which the request medical department belongs. The disease according to this embodiment is an example of the abnormality. The abnormality may be, for example, a disease, a congenital malformation, a congenital abnormality in a physical condition, an external injury, a physical disability or the like. The name of a disease is an example of information that specifies the type of an abnormality. Additionally, in place of the information that specifies the type of an abnormality, information that suggests the type of an abnormality may be used. The information that suggests the type of an abnormality is, for example, an expression that is described in the findings or impression of a diagnostic interpretation report and suggests a possibility that some abnormality exists in a specific part. In addition, the confirmation information CI according to this embodiment is an example of information concerning a referral letter to a decided handling destination. The information concerning a referral letter to a decided handling destination may be, for example, a referral letter generation screen. If the request, source that has requested an examination is net included in the decided handling destination, the processing circuitry 13 may display the referral letter generation screen on the display 193 without displaying the confirmation information CI.

As can be seen from these, according to the consultation support system 1, if a disease in which a medical department different from the request medical department specializes is described in the examination result, a suggestion to examine generation and issuance of a referral letter to the decided handling medical department can be presented to the doctor in charge in the request medical department, and omissions in necessary referral letter issuance can be reduced.

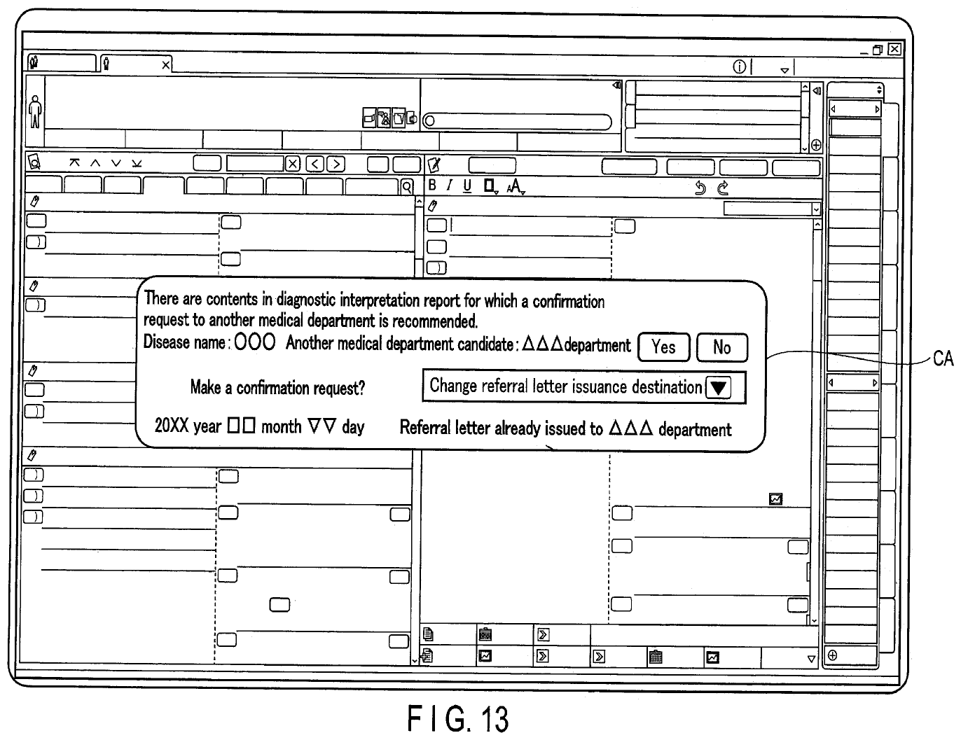

Additionally, according to the consultation support system 1, in a case in which the operator selects that generation of a referral letter is necessary for the confirmation information CI, a selection result representing that generation of a referral letter is necessary is associated with the examination result, and an input screen that enables input of a plurality of items in the referral letter is displayed on the display 193. In a case in which the operator selects that generation of a referral letter is unnecessary for the confirmation information CI, a selection result representing that generation of a referral letter is unnecessary can be associated with the examination result. This makes it possible to add a determination result concerning the necessity of generation of a referral letter to the decided handling medical department to a diagnostic interpretation report and discriminate the presence/absence of the determination concerning the necessity of generation of a referral letter. That is, according to the consultation support system 1, if a referral letter was issued to the decided handling medical department by the past examination for the same object, the date/time of issuance of the referral letter to the decided handling medical department can be displayed on the display 193 together with the decided handling medical department, as shown in FIGS. 7 and 13. It is therefore possible to reduce burden in determining the necessity of generation of a referral letter.

Additionally, according to the consultation support system 1, in a case in which the operator selects that generation of a referral letter is necessary for the confirmation information CI, it is possible to generate words concerning the disease and the decided handling medical department based on the analysis contents of the examination result and display, on the display 193, an input screen with the words in at least, one of a plurality of items in the referral letter.

In addition, according to the consultation support system 1, in a case in which the operator selects that generation of a referral letter is necessary for the confirmation information CI, the referral letter generated based on an instruction of the operator is output to the decided handling medical department, and a consultation concerning the disease in the examination result is executed for the object in the decided handling medical department, a flag regarding execution of the consultation can be set in the electronic medical record concerning the object.

As can be seen from these, according to the consultation support system 1, it is possible to reduce burden on the operator concerning generation or non-generation of a referral letter in accordance with the selection of necessity of generation of a referral letter to the decided handling medical department and improve the diagnosis efficiency.

According to the consultation support system 1, in a case in which selection in the confirmation information CI is not input for a predetermined period, the warning CA can be displayed on the display 193 of a terminal accessed by at least one of the doctor In charge of the object and a doctor belonging to the medical department to which the doctor in charge belongs. In addition, according to the consultation support system 1, in a case in which selection in the confirmation information is not input for a predetermined period, a warning can be displayed in the display screen of the display 193 when the electronic medical record concerning the object is accessed.

According to the consultation support system 1, it is possible to further store the period correspondence table associates a predetermined period with each of a plurality of diseases in accordance with the degree of exacerbation of each disease and decide the predetermined period based on the extracted disease name and the period correspondence table. Additionally, according to the consultation support system 1, in a case in which selection in the confirmation information is not input, and the doctor belonging to the decided handling medical department accesses the electronic medical record concerning the object, a notification representing that there is a possibility that a referral concerning the object is made to the decided handling medical department can be displayed on the display screen of the electronic medical record CEC concerning the object.

As can be seen from these, according to the consultation support system 1, even if the operator does not select the necessity of generation of a referral letter to the decided handling medical department, it is possible to support the decision about the necessity of generation of a referral letter at a timing appropriate for the doctor concerning the object.

As described above, according to the consultation support system 1 of this embodiment, since the necessity of generation of a referral letter to the decided handling medical department can be presented to the operator in accordance with the examination result, omissions in the issuance of referral letters can be reduced.

### First Modification

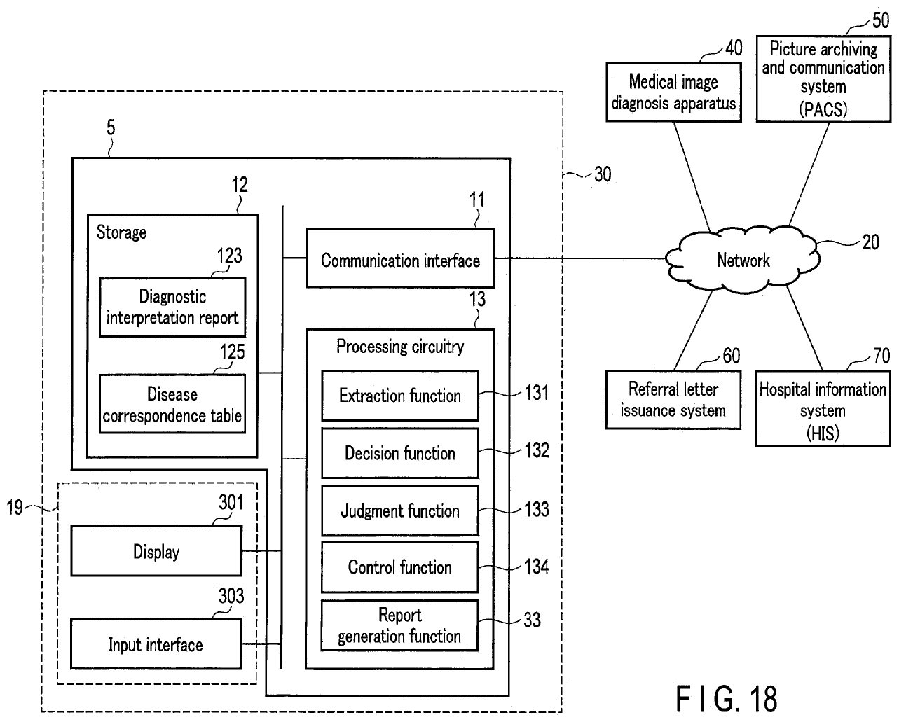

The first modification and the embodiment are different in that the consultation support system 1 is included in an RIS server. FIG. 18 is a block diagram showing the arrangements of the RIS 30 and an RIS server 5 including the consultation support system 1 and examples of an external apparatus and external systems connected to the RIS 30 via the network 20. Note that a display 301 and an input interface 303 in the RIS 30 shown in FIG. 18 may be connected as the input/output apparatus 19 via the network 20. Note that the processes of steps Sa6 to Sa13 in referral letter issuance support processing according to this modification may be executed in an HIS 70. The processing contents of the referral letter issuance support processing and the effects of this modification are similar to those in the description of the embodiment, and a description thereof will be omitted. Note that even a case in which the consultation support system 1 is included in the PACS 50 is similar to this modification, and a description thereof will be omitted.

### Second Modification

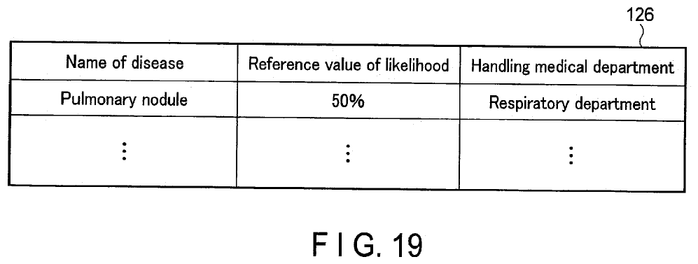

The second modification and the embodiment are different in that the presence/absence of display of the confirmation information CI is decided using a likelihood representing the degree of likelihood of a disease and the reference value of the likelihood. More specifically, the storage 12 further stores, in the disease correspondence table 125, the reference value of a likelihood representing the degree of likelihood of a disease in association with each of a plurality of diseases. FIG. 19 is a view showing an example of a disease correspondence table 126 according to this modification. As shown in FIG. 19, in the disease correspondence table 126, for example, if the disease name is pulmonary nodule, 50% is associated with the disease name as the reference value of the likelihood.

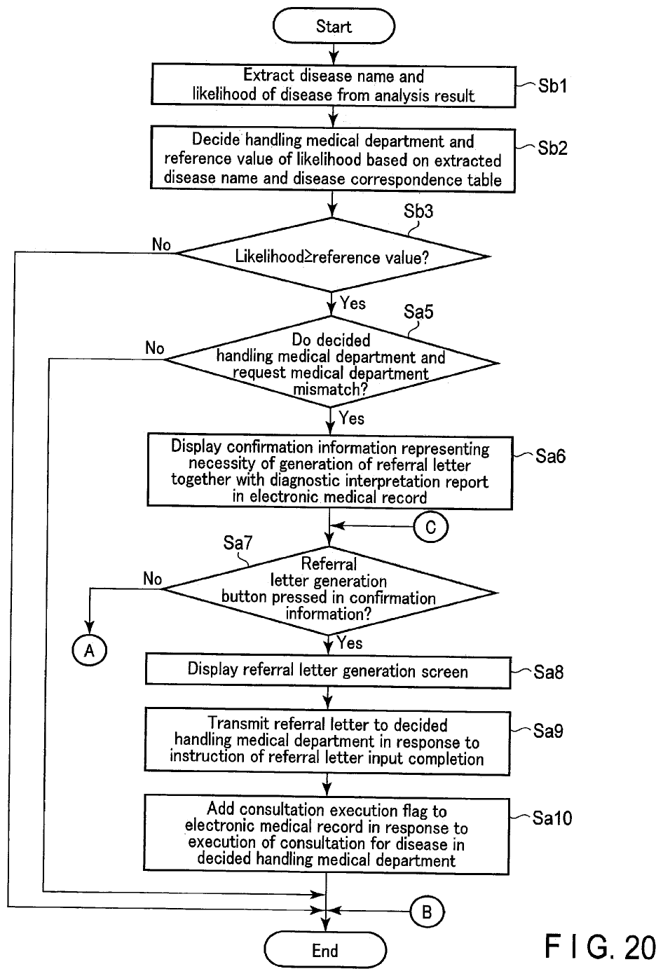

Referral letter issuance support processing according to this modification will be described below. FIG. 20 is a flowchart showing an example of the procedure of referral letter issuance support processing according to this modification. Processing of step Sb1 to be described below is executed next to step Sa2 shown in FIG. 4.

### Referral Letter Issuance Support Processing

(Step Sb1)

The processing circuitry 13 extracts a disease name and a likelihood from an analysis result by the extraction function 131. First, the processing circuitry 13 extracts a disease name from a structured report that is an analysis result, as in step Sa3. Next, the processing circuitry 13 extracts the likelihood of the disease described in the impression of a diagnostic interpretation report using a plurality of words and the disease name in the structured report.

(Step Sb2)

The processing circuitry 13 decides a handling medical department and the reference value of the likelihood by the decision function 132 based on the extracted disease name and the disease correspondence table. First, the processing circuitry 13 decides a handling medical department based on the extracted disease name and the disease correspondence table 126, as in step Sa4. Next, the processing circuitry 13 decides the reference value of the likelihood for the extracted disease name using the disease correspondence table 126 shown in FIG. 19 and the extracted disease name.

(Step Sb3)

The processing circuitry 13 judges, by the judgment function 133, whether the extracted likelihood is equal to or more than the reference value. More specifically, the processing circuitry 13 compares the value of the extracted likelihood with the reference value, thereby judging whether the likelihood is equal to or more than the reference value. If the likelihood is equal to or more than the reference value (YES in step Sb3), the processing circuitry 13 executes processing from step Sa5. For example, if the request medical department and the decided handling medical department do not match, and the extracted likelihood is equal to or more than the reference value, the processing circuitry 13 displays the confirmation information CI on the display 193. If the likelihood is not equal to or more than the reference value (NO in step Sb3), that is, if the likelihood is less then the reference value, the processing circuitry 13 ends the referral letter issuance support processing.

According to the above-described arrangement and operation, the following effects can be obtained in addition to the effects of this embodiment.

According to the consultation support system 1 of this modification, it is possible to further store the reference value of a likelihood representing the degree of likelihood of a disease in the disease correspondence table 126 in association with each of a plurality of diseases, further extract a likelihood corresponding to the extracted disease name from the analysis result, judge whether the extracted likelihood is equal to or more than the reference value, and if the request medical department and the decided handling medical department do not match, and the extracted likelihood is equal to or more than the reference value, display the confirmation information CI on the display 193.

Hence, according to the consultation support system 1, since the necessity of generation of a referral letter can be presented to the operator in accordance with the likelihood of the disease described in the examination result, it is possible to reduce burden on the operator while reducing omissions in the issuance of referral letters for examination results. In other words, it is possible to suppress excessive referral letter issuance proposals and improve the diagnosis efficiency.

### Third Modification

The third modification and the embodiment are different in that in addition to the item concerning the necessity of generation of a referral letter, confirmation information further includes a next time confirmation item for selecting confirmation of the necessity at the next time of consultation for the object, and if the next time confirmation item is selected by the operator, the confirmation information is displayed on the display screen of the electronic medical record concerning the object at the next time of consultation.

The storage 12 stores, as the display form of the confirmation information, the next time confirmation item that enables selection of confirmation of the necessity at the next time of consultation for the object in addition to the item concerning the necessity of generation of a referral letter.

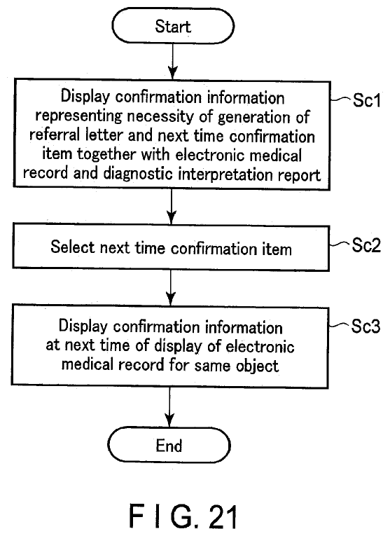

Referral letter issuance support processing according to this modification will be described below. FIG. 21 is a flowchart showing an example of the procedure of referral letter issuance support processing. Processing of step Sc1 to be described below is executed next to step Sa5 shown in FIG. 21.

### Referral Letter Issuance Support Processing

(Step Sc1)

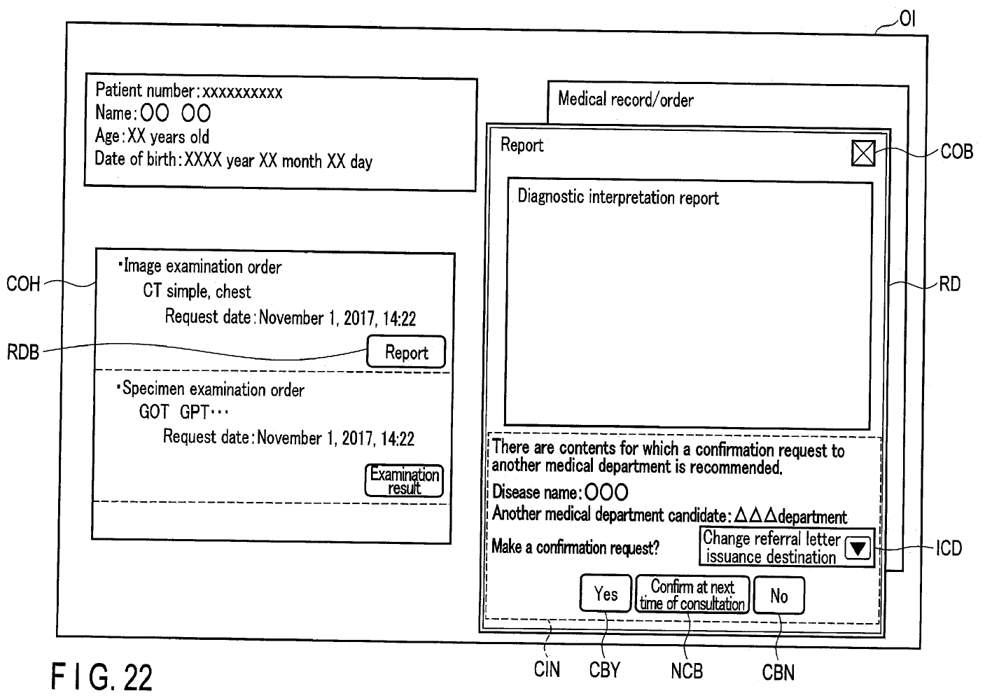

The processing circuitry 13 displays confirmation information that shows the necessity of generation of a referral letter from the request medical department to the decided handling medical department and the next time confirmation item on the display 193 by the control function 134 together with a diagnostic interpretation report. FIG. 22 is a view showing an example of confirmation information CIN displayed on the display 193 in this step. As shown in FIG. 22, when the operator presses the report display button RDB in the medical record/order history display region COH of the order input screen OI, the processing circuitry 13 displays the confirmation information CIN on the display 193 together with the diagnostic interpretation report RD. The confirmation information CIN includes, for example, “There are contents for which a confirmation request to another medical department is recommended. Disease name: ◯◯◯, Another medical department candidate: ΔΔΔ department. Make a confirmation request?”, the referral letter generation button CBY, the referral letter non-generation button CBN, the issuance destination change button ICD, and a button (to be referred to as a next time confirmation button hereinafter) NCB used to decide the next time confirmation item.

(Step Sc2)

The next time confirmation item is selected based on an instruction of the operator via the input interface 191. That is, the next time confirmation button NCB is pressed based on an instruction of the operator via the input interface 191 without pressing the referral letter generation button CBY and the referral letter non-generation button CBN. Note that if the referral letter generation button CBY or the referral letter non-generation button CBN is pressed before the processing of this step, the processing circuitry 13 ends the referral letter issuance support processing.

(Step Sc3)

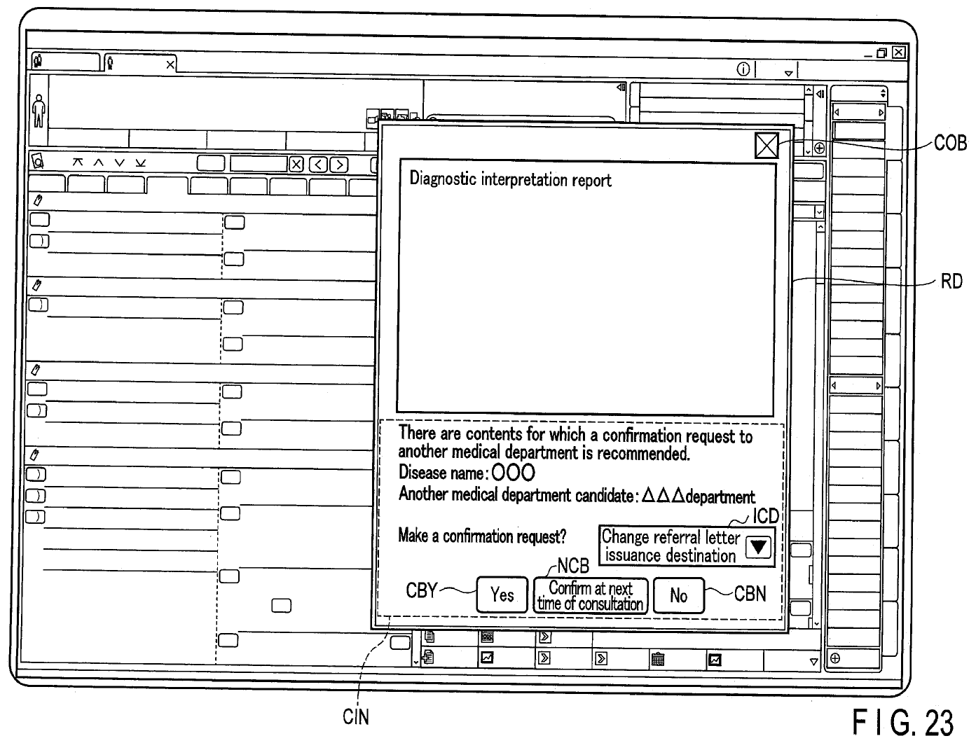

At the next time of display of the electronic medical record for the same object, that is, at the next time of consultation, the processing circuitry 13 displays the confirmation information CIN on the display 193 by the control function 134. FIG. 23 is a view showing an example of the confirmation information CIN displayed on the display screen of the electronic medical record concerning the object at the next time of consultation. As shown in FIG. 23, the confirmation information CIN is displayed on the display screen of the electronic medical record. Subsequent processing is similar to that from step Sa7 of the flowchart shown in FIG. 4, and a description thereof will be omitted.

According to the above-described arrangement and operation, the following effects can foe obtained in addition to the effects of this embodiment.

According to the consultation support system 1 of this modification, in addition to the item concerning the necessity, the confirmation information CIN further includes the next time confirmation item for selecting confirmation of the necessity at the next time of consultation for the object, and if the next time confirmation item NCB is selected by the operator, the confirmation information CIN can be displayed on the display screen of the electronic medical record concerning the object at the next time of consultation.

Hence, according to the consultation support system 1, since selection of the necessity of generation of a referral letter can temporarily be held while preventing omissions in the issuance of referral letters for examination results, it is possible to improve the diagnosis efficiency while reducing burden on the operator.

### Fourth Modification

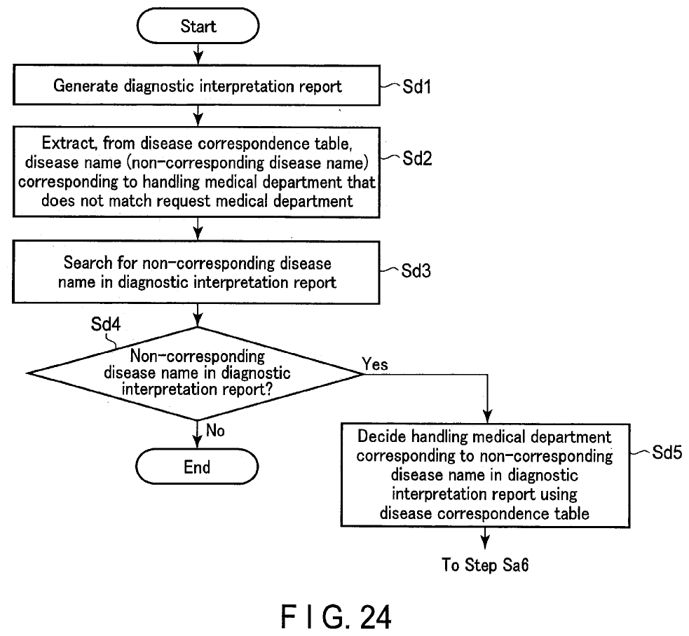

The fourth modification and the embodiment are different in that a disease name corresponding to a handling medical department that does not match the request medical department is extracted from the disease correspondence table, and if the extracted disease name exists in a diagnostic interpretation report, confirmation information is displayed on the display 193. Referral letter issuance support processing according to this modification will be described with reference to FIG. 24. FIG. 24 is a flowchart showing an example of the procedure of referral letter issuance support processing according to this modification. Step Sd1 is similar to the processing of step Sa1, and a description thereof will be omitted.

(Step Sd2)

The processing circuitry 13 extracts the name of a disease that is not associated with the request medical department that has requested an examination concerning the object from the disease correspondence table by the extraction function 131. More, specifically, the processing circuitry 13 specifies, in the disease correspondence table, handling medical departments that do not match the request medical department, that is, a plurality of handling medical departments different from the request medical department. Next, the processing circuitry 13 extracts a plurality of disease names corresponding to the plurality of specified handling medical departments (to be referred to as specified handling medical departments hereinafter) from the disease correspondence table. With these processes, the processing circuitry 13 extracts the names of diseases (to be referred to as non-corresponding disease names hereinafter) that are not associated with the request medical department from the disease correspondence table.

(Step Sd3)

The processing circuitry 13 searches for a non-corresponding disease name in the diagnostic interpretation report by the judgment function 133. For example, the processing circuitry 13 searches for a non-corresponding disease name in the described contents of the findings of the diagnostic interpretation report.

(Step Sd4)

The processing circuitry 13 judges the presence/absence of an extracted disease name by the judgment function 133 based on the search result in step Sd3. More specifically, the processing circuitry 13 judges the presence/absence of a non-corresponding disease name in the diagnostic interpretation report. For example, the processing circuitry 13 judges whether a non-corresponding disease name is included in the described contents of the findings of the diagnostic interpretation report. If a non-corresponding disease name exists in the diagnostic interpretation report (YES in step Sd4), the processing circuitry 13 executes processing of step Sd5. If a non-corresponding disease name, does not exist in the diagnostic interpretation report (NO in step Sd4), the processing circuitry 13 ends the referral letter issuance support processing.

(Step Sd5)

The processing circuitry 13 decides a handling medical department corresponding to the extracted disease name existed in the examination result by the decision function 132 using the disease correspondence table. That is, the processing circuitry 13 decides a handling medical department (decided handling medical department) corresponding to the non-corresponding disease name existed in the diagnostic interpretation report. After the processing of step Sd5, the processing circuitry 13 executes processing from step Sa6. Processing from step Sa6 is similar to that of the embodiment, and a description thereof will be omitted.

According to the above-described arrangement and operation, the following effects can be obtained in addition to the effects of this embodiment.

According to the consultation support system 1 of this modification, it is possible to store the disease correspondence table in which a plurality of handling medical departments capable of handling a plurality of diseases are associated with the names of the plurality of diseases, extract the name of a disease that is not associated with the request medical department that has requested the examination concerning the object from the disease correspondence table, search for the extracted disease name in the examination result to judge the presence/absence of the extracted disease name in the examination result, decide a handling medical department corresponding to the extracted disease name in the examination result using the disease correspondence table, and display, on the display 193, the confirmation information CI representing the necessity of generation of a referral letter from the request medical department to the decided handling medical department.

That is, according to the consultation support system 1 of this modification, it is possible to store the correspondence table in which handling destinations capable of making a consultation are associated with a plurality of abnormalities, extract, from the plurality of abnormalities included in the correspondence table, an abnormality with which the request medical department that has requested the examination concerning the object is not associated as a handling destination, and if information that specifies or suggests the type of the extracted abnormality is included in the examination result, display, on the display 193, information concerning a referral letter to a handling destination capable of making a consultation about the extracted abnormality.

Hence, according to the consultation support system 1, since the decided handling medical department can more easily be acquired without executing natural language processing for the diagnostic interpretation report, implementation is easy. In addition, since natural language processing is not executed, the processing time needed for the referral letter issuance support processing can be shortened, and the diagnosis efficiency can further be improved. As an application example of this modification, the first modification and the third modification may be applied to this modification.

As an application example of this embodiment and the first to fourth modifications, if the technical idea of the consultation support system 1 is implemented by a cloud or the like, a server on the Internet includes, for example, the storage 12 and the processing circuitry 13 in the block diagram of FIG. 1. At this time, referral letter issuance support processing is implemented by installing, in the processing circuitry 13, a program configured to execute the processing and executing the installed program. In addition, the input interface 191 and the display 193 are implemented on, for example, various kinds of terminals via the Internet.

### Other Modifications

In another modification, the processing circuitry 13 extracts a disease name from a diagnostic interpretation report, extracts the names of diseases for which the request medical department can make a consultation from a disease correspondence table in which a disease for which each medical department can make a consultation is associated with the medical department, and judges whether the disease extracted from the diagnostic interpretation report is included in the diseases for which the request medical department can make a consultation. Then, if the disease extracted from the diagnostic interpretation report is not included in the diseases for which the request medical department can make a consultation, the processing circuitry 13 decides a medical department capable of making a consultation for the disease extracted from the diagnostic interpretation report, and displays, on the display, confirmation information concerning the necessity of generation of a referral letter to the decided medical department (handling medical department).

Hence, according to the consultation support system 1, it is possible to extract information that specifies or suggests the type of an abnormality in the examination result from an analysis result obtained by analyzing the result of an examination concerning the object, decide an abnormality concerning the object using the extracted information that specifies or suggests the type of the abnormality, and if the decided abnormality is not included in the abnormalities for which the request source that has requested the examination concerning the object can make a consultation, display, on the display, information concerning a referral letter to the handling destination.

According to the consultation support system 1 of the above-described embodiment, modifications, and the like, it is possible to reduce omissions in the issuance of referral letters for examination results. That is, according to the consultation support system 1, a disease name pointed out in a report corresponding to an examination result such as a diagnostic interpretation report is extracted, and if a medical department capable of handling a disease corresponding to the extracted disease name does not match the request medical department, the necessity of generation of a referral letter is proposed to the doctor in charge of the object, thereby reducing omissions in the issuance of referral letters or an oversight of the issuance of referral letters.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A consultation support system comprising:
processing circuitry configured to
extract information that specifies or suggests a type of an abnormality in a result of an examination concerning an object from an analysis result obtained by analyzing the result of the examination,
decide a handling destination capable of making a consultation about the abnormality specified, or suggested in the analysis result using the extracted information that specifies or suggests the type of the abnormality, and
display information concerning a referral letter to the decided handling destination on a display if a request source that has requested the examination .is not included in the decided handling destination.

2. The consultation support system according to claim 1, wherein the processing circuitry is further configured to judge whether the request source is included in the handling destination by comparing the request source and the handling destination.

3. The consultation support system according to claim 2, wherein the processing circuitry is further configured to
further extract a likelihood representing a degree of likelihood of the specified or suggested abnormality from the analysis result.
judge whether the extracted likelihood is equal to or more than a reference value, and
display the information concerning the referral letter on the display if the request source is not included in the decided handling destination, and the extracted likelihood is equal to or more than the reference value.

4. The consultation support system according to claim 1, wherein the processing circuitry is further configured to acquire the analysis result that enables extraction of the information that specifies or suggests the type of the abnormality by analyzing whether the information that specifies or suggests the type of the abnormality is included in the result of the examination.

5. The consultation support system according to claim 1, wherein the information concerning the referral letter is confirmation information representing necessity of generation of a referral letter from the request source to the decided handling destination or a generation screen of a referral letter.

6. The consultation support system according to claim 1, wherein the information concerning the referral letter is confirmation information representing necessity of generation of a referral letter from the request source to the decided handling destination, and
the processing circuitry is further configured to
necessary for the confirmation information associate a selection result by an operator with the result of the examination and display an input screen that enables input of a plurality of items in the referral letter on the display if the operator selects that generation of the referral letter is necessary for the confirmation information, and
necessary for the confirmation information associate the selection result by the operator with the result of the examination if the operator selects that generation of the referral letter is unnecessary for the confirmation information.

7. The consultation support system according to claim 6, wherein the processing circuitry is further configured to
necessary for the confirmation information generate words concerning the abnormality and the decided handling destination based on analysis contents of the result of. the examination if the operator selects that generation of the referral letter is necessary for the confirmation information, and
display the input screen with the words in at least one item of the items in the referral letter on the display.

8. The consultation support system according to claim 1, wherein the information concerning the referral letter is confirmation information representing necessity of generation of a referral letter from the request source to the decided handling destination, and
the processing circuitry is further configured to set a flag regarding execution of a consultation in an electronic medical record concerning the object if an operator selects that generation of the referral letter is necessary for the confirmation information, the referral letter generated based on an instruction of the operator is output to the decided handling destination, and the consultation concerning the abnormality in the result of the examination is executed for the object in the decided handling destination.

9. The consultation support system according to claim 1, wherein the information concerning the referral letter is confirmation information representing necessity of generation of a referral letter from the request source to the decided handling destination,
the confirmation information further includes a next time confirmation item for selecting the necessity at the next time of consultation for the object in addition to an item concerning the necessity, and
the processing circuitry is further configured to display the confirmation information on a display screen of an electronic medical record concerning the object at the next time of consultation, if the operator selects the next time confirmation item.

10. The consultation support system according to claim 1, wherein the information concerning the referral letter is confirmation information representing necessity of generation of a referral letter from the request source to the decided handling destination, and
the processing circuitry is further configured to display a warning on a display of a terminal accessed by at least one of a doctor in charge of the object and a doctor belonging to a medical department to which the doctor in charge of the object belongs if selection for the confirmation information is not input for a predetermined period.

11. The consultation support system according to claim 1, wherein the information concerning the referral letter is confirmation information representing necessity of generation of a referral letter from the request source to the decided handling destination, and
the processing circuitry is further configured to display a warning on a display screen of an electronic medical record concerning the object when the electronic medical record is accessed if selection for the confirmation information is not input for a predetermined period.

12. The consultation support system according to claim 10, wherein the processing circuitry is further configured to decide the predetermined period in accordance with a degree of exacerbation of the specified or suggested abnormality.

13. The consultation support system according to claim 1, wherein the information concerning the referral letter is confirmation information representing necessity of generation of a referral letter from the request source to the decided handling destination, and
the processing circuitry is further configured to display a notification representing that there is a possibility that a referral concerning the object is made to the decided handling destination on a display screen of an electronic medical record concerning the object if selection for the confirmation information is not input and a doctor belonging to the decided handling destination accesses the electronic medical record concerning the object.

14. A consultation support system comprising:
a memory configured to store a correspondence table in which a handling destination capable of making a consultation is associated with each of a plurality of abnormalities; and
processing circuitry configured to
extract an abnormality with which a request destination that has requested an examination concerning an object is net associated as the handling destination from the plurality of abnormalities included in the correspondence table, and
display information concerning a referral letter to a handling destination capable of making a consultation about the extracted abnormality on a display if information that specifies or suggests a type of the extracted abnormality is included in a result of the examination.

15. A consultation support system comprising:
processing circuitry configured to
extract information that specifies or suggests a type of an abnormality in a result of an examination from an analysis result obtained by analyzing the result of the examination concerning an object, and
display information concerning a referral letter to a handling destination capable of making a consultation about the abnormality specified or suggested in the analysis result using the information that specifies or suggests the type of the extracted abnormality on a display if the abnormality specified or suggested in the analysis result is not included in abnormalities for which a request source can make a consultation,.
